(19) United States
(12) Patent Application Publication
(10) Pub. No.: US 2009/0012018 A1
(43) Pub. Date: Jan. 8, 2009

(54) Inhibition of pancretic cancer cell growth

(76) Inventors: Matthias Hebrok, Belmont, CA (US); Marina Pasca Di Magliano, San Francisco, CA (US)

Correspondence Address: Medlen& Carroll LLP, 101 Howard Street Suite 350, San Francisco, CA, 94105, US

(21) Appl. No.: 11/662,853

(22) PCT Filed: Sep. 13, 2005
(86) PCT No.: PCT/US05/32950

§ 371 Date: Mar. 27, 2008

(30) Foreign Application Priority Data
Sep. 13, 2004 (US) 60/609693

(51) Int. Cl. A61K 31/7068; C12N 5/06; C12N 15/63; A61K 31/59; A61P 35/00; A61K 31/7105; A61K 31/192; C12N 5/08; A61K 31/505

(52) U.S. Cl. 514/44; 435/375; 435/455; 435/372; 514/274; 514/49; 514/167; 514/569

(57) ABSTRACT

The present invention relates to compositions and methods for inhibition of signal transduction pathways in cancer cells. In particular, the present invention provides methods and compositions comprising Wnt and Hedgehog pathway inhibitors for reducing proliferation of adenocarcinoma cells.

The invention was made in part with Government support from the National Institutes of Health, Grants DK60533 and DK60694. As such, the Government has certain rights in the invention.

### FIELD OF THE INVENTION

The present invention relates to compositions and methods for inhibition of signal transduction pathways in cancer cells. In particular, the present invention provides methods and compositions comprising Wnt and Hedgehog pathway inhibitors for reducing proliferation of adenocarcinoma cells.

### BACKGROUND OF THE INVENTION

Pancreatic cancer ranks just below lung cancer, colon cancer and breast cancer, as the leading cause of cancer death in the United States. The prognosis is poor, with a five-year survival rate of less than one percent, and a median survival time post-diagnosis of around six months. Early detection of pancreatic cancer is difficult, and over 80% of patients are found to have inoperable disease (Li et al., Lancet, 363:1049-1057, 2004), due to invasion of vital structures or metastasis. For locally advanced, unresectable, and metastatic disease, treatment is primarily palliative, although chemotherapy and radiation therapy may also be offered.

The most frequently employed chemotherapies for pancreatic cancer are the fluoropyrimidines 5-fluorouracil and 2′,2′-difluorodeoxycytidine (gemcitabine hydrochloride or GEMZAR). These drugs induce apoptosis of cancer cells by inhibiting DNA replication. Frequent side effects of these medications include low blood counts, nausea and vomiting. In addition, although chemoradiation treatment regimens permit a small minority of patients to become surgical candidates, chemotherapy generally only extends the survival time of pancreatic cancer patients by about six months.

Thus, what is needed in the art are more effective therapy regimens for treating pancreatic cancer. In particular, it would be desirable to have treatment options based on the molecular biology of pancreatic cancer.

### SUMMARY OF THE INVENTION

The present invention relates to compositions and methods for inhibition of signal transduction pathways in cancer cells. In particular, the present invention provides methods and compositions comprising Wnt and Hedgehog pathway inhibitors for reducing proliferation of adenocarcinoma cells.

Specifically, the present invention provides methods comprising contacting a pancreatic adenocarcinoma cell with both a Hedgehog pathway inhibitor and a Wnt pathway inhibitor, under conditions suitable for retarding growth of the pancreatic adenocarcinoma cell. In some embodiments, the pancreatic adenocarcinoma cell is a human cell. In some preferred embodiments, the retarding growth comprises inhibiting proliferation of the pancreatic adenocarcinoma cell, and/or inducing death of the pancreatic adenocarcinoma cell. In especially preferred embodiments, the inducing death of the pancreatic adenocarcinoma cell is essentially unaccompanied by death of untransformed pancreatic ductal cells. In exemplary embodiments, the Hedgehog pathway inhibitor is cyclopamine or Gli (e.g., Gli1 and/or Gli2) siRNA. The present invention also provides embodiments in which the target of the Hedgehog pathway inhibitor is selected from but not limited to Smoothened, Sonic Hedgehog, Desert Hedgehog, Indian Hedgehog Gli1 and Gli2. In additional exemplary embodiments, the Wnt pathway inhibitor is selected from but not limited to sulindac, sulindac sulfide, sulindac sulfone, ICAT, dnLef1, and β-catenin siRNA. The present invention also provides embodiments in which the Wnt pathway inhibitor is selected from but not limited to LRP, Frizzled, Wnt, Dishevelled, β-catenin, Lef and Tcf. In some preferred embodiments, the methods further comprise assessing Wnt pathway activity in the adenocarcinoma cell by examining nuclear or cytoplasmic accumulation of β-catenin, prior to the contacting step.

Furthermore the present invention provides methods of treating cancer of the pancreas, comprising: providing a Hedgehog pathway inhibitor and a Wnt pathway inhibitor; and administering the Hedgehog pathway inhibitor and the Wnt pathway inhibitor to a subject diagnosed with cancer of the pancreas, under conditions suitable for retarding growth of the cancer. In some embodiments the subject is a human. In particularly preferred embodiments, the cancer of the pancreas is pancreatic adenocarcinoma. The present invention provides methods wherein the retarding growth of the cancer comprises inhibiting proliferation of at least one cell of the pancreatic adenocarcinoma and/or inducing death of at least one cell of the pancreatic adenocarcinoma. In preferred embodiments, the inducing death of at least one cell of the pancreatic adenocarcinoma is essentially unaccompanied by death of untransformed pancreatic ductal cells. In other preferred embodiments, the retarding growth of the cancer comprises reducing volume of the pancreatic adenocarcinoma. In some embodiments, the Hedgehog pathway inhibitor and the Wnt pathway inhibitor are each formulated in a pharmaceutically acceptable carrier. The present invention also provides treatment methods further comprising administering a standard treatment regimen for cancer of the pancreas, to the subject. In some embodiments, the standard treatment regimen comprises one or more of resection, radiation and chemotherapy. In a subset of these embodiments, the chemotherapy is selected from the group consisting of 5-fluorouracil, and 2′,2′-difluorodeoxycytidine. In addition, the present invention provides treatment methods further comprising administering an adjunct treatment regimen for cancer of the pancreas, to the subject. In some embodiments, the adjunct treatment regimen is selected from the group consisting of a platinum analogue, topoisomerase-inhibitor, antimicrotubule agent, proteosome inhibitor, vitamin D analogue, arachidonic acid pathway inhibitor, histone deacytylator inhibitor, farnesyltransferase inhibitor and epidermal growth factor-based therapy.

Moreover, the present invention provides kits for treating pancreatic adenocarcinoma in a subject, comprising: a) a Hedgehog pathway inhibitor; b) a Wnt pathway inhibitor; and c) instructions for using the Hedgehog pathway inhibitor and the Wnt pathway inhibitor to treat pancreatic adenocarcinoma in a subject. In some embodiments, the Hedgehog pathway inhibitor is cyclopamine. In further embodiments, the Wnt pathway inhibitor is selected from but not limited to sulindac, sulindac sulfide, sulindac sulfone, ICAT, dnLef1, and β-catenin siRNA.

### DESCRIPTION OF THE FIGURES

FIG. 1 illustrates that Wnt signaling is active in human pancreatic adenocarcinoma cells. Panel A provides an immunofluorescent analysis of β-catenin localization in normal human pancreas (control) and in pancreatic adenocarcinoma samples (tumor 1-3). β-catenin expression is confined to the cell membrane of controls, while cytoplasmic and nuclear accumulation of β-catenin is observed in a subset of human tumors (3/7). Panel B provides an RT-PCR analysis of components of the Wnt signaling pathway in 26 human pancreatic adenocarcinoma cell lines: 1-MiaPaca2; 2-Panc1; 3-CFPAC1; 4-HPAFII; 5-Capan-2; 6-AsPC1; 7-Hs766T; 8-BxPC3; 9-COLO357; 10-L3.3; 11-L3.6sl; 12-L3.6 pl; 13-SW1990; 14-SU86.86; 15-PL45; 16-HPAC; 17-MPanc96; 18-Panc1.28; 19-Panc2.03; 20-Panc2.13; 21-Panc3.27; 22-Panc4.21; 23-Panc5.04; 24-Panc6.03; 25-Panc8.13; 26-Panc10.05. Panel C provides a schematic of the Fop/Top-Flash reporter assay. Panel D is a graph indicating that Wnt signaling is active in all nine pancreatic adenocarcinoma cell lines tested. The columns on the right depict activation of the Top-Flash reporter, while the columns on the left depict basal activity of the Fop-Flash. Expression of non-phosphorylated β-catenin was observed in four pancreatic cancer cell lines (CFPAC, BxPC3, L3.6sl and Panc4.21) by western blot analysis.

FIG. 2 illustrates that inhibition of Wnt signaling blocks adenocarcinoma cell proliferation. Panel A graphically depicts the level of Wnt signaling observed in four pancreatic adenocarcinoma cell lines transfected with Fop-Flash or Top-Flash, and co-transfected with an expression vector containing the Wnt-inhibitors Icat; and co-transfected with a dominant-negative Lef1 (Lef-dn). Cells were harvested 24 hours after transfection. Reduction of Top-Flash activity indicates inhibition of Wnt signaling in pancreatic cancer cells. P-values were calculated in comparison to Top-Flash activity. Panel B provides images of pancreatic adenocarcinoma cells stained with an anti-β-catenin antibody after being transfected with a siRNA directed against β-catenin. The levels of β-catenin protein decreased in a dose-dependent manner, while control siRNA transfected cells have β-catenin protein levels indistinguishable from the untransfected cells (untreated). Panel C illustrates that transfection with an Icat-IRES-eGFP expression vector (Icat) or dominant-negative Lef-IRES-eGFP (dnLef) strongly inhibits growth of four pancreatic cancer cell lines, measured as the ability to incorporate BrdU. Control cells were transfected with an empty IRES-eGFP expression vector. All cells were harvested 48 hours after transfection. P values are shown in comparison to transfected controls. Panel D illustrates that proliferation is reduced in a dose-dependent manner in cells treated for 48 hrs with anti-β-catenin siRNA (100 nM or 300 nM) as compared to control cells (untransfected or transfected with 300 mM control siRNA). P values are shown in comparison to untreated controls. Panel E graphically depicts the levels of apoptosis (measured as cells with DNA content lower than the diploid amount) in control transfected cells and in cells transfected with Icat or dominant-negative Lef. Panel F illustrates that treatment of pancreatic cancer cells with anti-β-catenin siRNA increases apoptosis in a dose dependent manner. Error bars are shown as SD, and P-values equal: #, not significant; *p<0.05; or **p<0.01.

FIG. 3 illustrates that inhibition of Wnt signaling reduces xenograft tumor growth in nude mice. Panel A illustrates that sulindac and sulindac sulfone inhibit Wnt activity in four pancreatic adenocarcinoma cells. Cells were transfected with Fop-Flash or Top-Flash and grown in control medium or medium containing 250 μM sulindac or 150 μM sulindac sulfone. Reduction of Top-Flash activity indicates inhibition of Wnt signal transduction in pancreatic cancer cells. Panel B illustrates that treatment with sulindac and sulindac sulfone strongly inhibits growth of BxPC3, L3.6sl and Panc4.21 cell lines as compared to untreated controls. CFPAC cells are not affected by sulindac treatment and only mildly affected by treatment with sulindac sulfone. Cells treated with COX-2 inhibitors NS398 and sodium salicylate show comparable growth to untreated control cells. Panel C illustrates that growth of BxPC3 and L3.6sl tumors is strongly reduced in mice treated with sulindac for five days as compared to untreated controls, while tumors derived from the marginally responsive cell line CFPAC1 are largely unaffected, while Panel D provides a comparison of tumor weights after control (left) and sulindac treatment (right), with n=4. Panel E illustrates that growth of BxPC3 and L3.6sl tumors is reduced in mice treated with sulindac sulfone for five days as compared to untreated controls. Again, tumors derived from the CFPAC1 cell line are unaffected. Panel F provides a comparison of tumor weights after control (left) and sulindac sulfone treatment (red), with n=7. Error bars are shown as SD, and P-values equal: #, not significant; *p<0.05; or **p<0.01.

FIG. 4 illustrates that Hedgehog signaling regulates Wnt activity in untransformed duct and pancreatic adenocarcinoma cells. Panel A illustrates that activity of the Top-Flash vector in pancreatic adenocarcinoma cells (columns 2) is inhibited in response to sulindac treatment (columns 3), as well as in response to cyclopamine (columns 4), an inhibitor of hedgehog signaling pathway. Panel B illustrates that activity of the hedgehog pathway, measured with a Gli-binding site-luciferase (GliBS, columns 2) construct, is inhibited by cyclopamine (columns 4) but is unaffected by sulindac treatment (columns 3). Panel C provides immunofluorescence images of pancreatic cancer cells grown in control conditions (top) or treated with cyclopamine for 24 hrs (bottom). Cyclopamine treatment inhibits Tcf4 and Tcf1 expression. Panel D provides immunofluorescence images of PDC cells illustrating that Hedgehog pathway activation by transfection of PDC cells with a myc-tagged-version of the constitutively active form of Gli2 (Gli2ΔN2) leads to upregulation of Tcf expression. Panel E graphically illustrates that activation of the Hedgehog pathway in two PDC=Gli2ΔN2 clones (#1 and #2) results in a significant increase in Wnt signaling. Panel F graphically illustrates that activation of the Wnt pathway by transfection of PDC cells with a dominant active form of Lef1, does not affect the level of Hedgehog signaling. Panel G illustrates that constitutive activation of the Wnt pathway induces colony formation in PDC-Lef-da cells, whereas wild type PDCs are unable to form colonies in soft agar. Panel H graphically illustrates the effect of treatment of pancreatic cancer cells with 75 μM sulindac sulfone (columns 2), 5 μM cyclopamine (columns 3) or both 75 μM sulindac sulfone and 5 μM cyclopamine (columns 4) inhibits cell proliferation as measured by BrdU incorporation. The combined treatment results in increased reduction of cell proliferation when compared to treatment with individual inhibitors. Panel I provides phase contrast images of pancreatic cancer cell lines treated with cyclopamine (5 μM) and β-catenin siRNA (100 nM). The combined treatment inhibits cell proliferation and induces cell death much more effectively than treatment with individual inhibitors. Error bars are shown as SD, and P-values equal: #, not significant; *p<0.05; or **p<0.01.

FIG. 5 provides phase contrast images of pancreatic cancer cell lines treated with increasing concentrations of sulindac in panel A, and sulindac sulfone in panel B.

FIG. 6 illustrates that combined treatment of cells with sulindac sulfone and cyclopamine is not generally cytotoxic. As shown in the phase contrast images, two pancreatic cancer cell lines (Panc1.28 and Panc 10.05) that are not sensitive to cyclopamine and sulindac sulfone alone, as well as non-transformed mouse pancreatic duct cells (PDCs) are unaffected by combined treatment with cyclopamine and sulindac sulfone.

FIG. 7 illustrates the effect of combined inhibition of Wnt and Hedgehog pathways in pancreatic cancer cells. Panel A provides phase contrast images of cells that were transfected with control siRNA (300 nM), β-catenin siRNA (150 nM, plus control siRNA 150 nM), GLI siRNA (GLI1 si RNA, 75 nM plus GLI2 siRNA, 75 nM plus control siRNA 150 nM), or with a combination of β-catenin and GLI si RNA (β-catenin 150 nM, GLI1 75 nM and GLI2 75 nM). Cell growth was reduced following β-catenin or GLI siRNA transfection. Increased inhibition of proliferation was observed in the cells in which both the Wnt and the Hedgehog pathways were targeted. Panel B provides immunofluorescent images of pancreatic cancer cells stained with an anti-cleaved caspase 3 antibody indicating that inhibition of Wnt and Hedgehog pathways induces cell apoptosis. The cells were incubated for 48 hrs prior to harvesting. Panel C provides a graph of the four cells lines after analysis by flow cytometry following siRNA transfection (48 hrs incubation) and staining with an anti-cleaved caspase 3 antibody to quantify the number of apoptotic cells. Cells were transfected with a control siRNA (columns 1), anti-β-catenin siRNA (columns 2), anti-GLI1 and GLI2 siRNA (columns 3) and a combination of β-catenin and GLI1/GLI2 (columns 4). The P-values are calculated in comparison with the cells transfected with control siRNA; #, not significant; **, p<0.01.

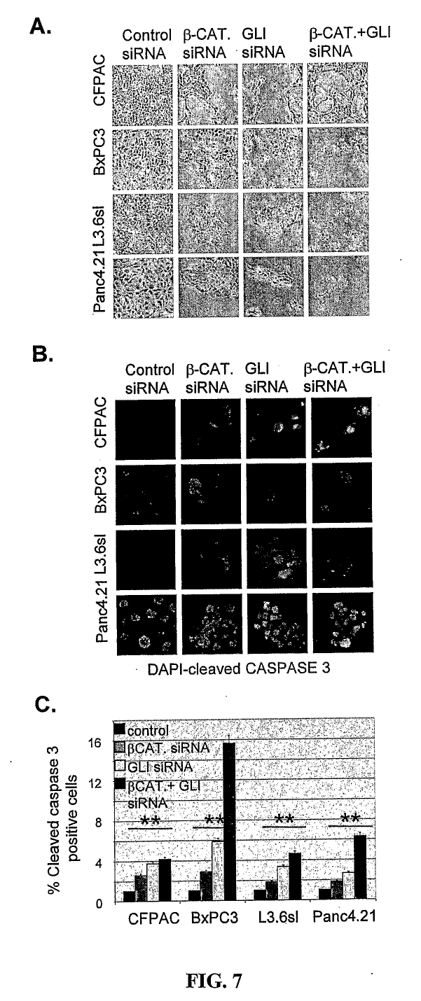

### DEFINITIONS

To facilitate an understanding of the present invention, a number of terms and phrases are defined below:

The term “gene” refers to a nucleic acid (e.g., DNA) sequence that comprises coding sequences necessary for the production of a polypeptide or precursor or RNA (e.g., tRNA, siRNA, rRNA, etc.). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends, such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region, which may be interrupted with non-coding sequences termed “introns” or “intervening regions” or “intervening sequences.” Introns are removed or “spliced out” from the nuclear or primary transcript, and are therefore absent in the messenger RNA (mRNA) transcript. The mRNA functions during translation to specify the sequence or order of amino acids in a nascent polypeptide.

In particular, the term “β-catenin” refers to the full-length β-catenin nucleotide sequence. However, it is also intended that the term encompass fragments of the β-catenin nucleotide sequence, as well as other domains (e.g., functional domains) within the full-length β-catenin nucleotide sequence. Furthermore, the terms “β-catenin gene,” “P-catenin nucleotide sequence,” and “β-catenin polynucleotide sequence” encompass DNA, cDNA, and RNA sequences.

The term “plasmid” as used herein, refers to a small, independently replicating, piece of DNA. Similarly, the term “naked plasmid” refers to plasmid DNA devoid of extraneous material typically used to affect transfection. As used herein, a “naked plasmid” refers to a plasmid substantially free of calcium phosphate, DEAE-dextran, liposomes, and/or polyamines.

As used herein, the term “purified” refers to molecules (polynucleotides or polypeptides) that are removed from their natural environment, isolated or separated. “Substantially purified” molecules are at least 50% free, preferably at least 75% free, and more preferably at least 90% free from other components with which they are naturally associated.

The term “recombinant DNA” refers to a DNA molecule that is comprised of segments of DNA joined together by means of molecular biology techniques. Similarly, the term “recombinant protein” refers to a protein molecule that is expressed from recombinant DNA.

The term “fusion protein” as used herein refers to a protein formed by expression of a hybrid gene made by combining two gene sequences. Typically this is accomplished by cloning a cDNA into an expression vector in frame with an existing gene. The fusion partner may act as a reporter (e.g., βgal) or may provide a tool for isolation purposes (e.g., GST).

Suitable systems for production of recombinant proteins include but are not limited to prokaryotic (e.g., Escherichia coli), yeast (e.g., Saccaromyces cerevisiae), insect (e.g., baculovirus), mammalian (e.g., Chinese hamster ovary), plant (e.g., safflower), and cell-free systems (e.g., rabbit reticulocyte).

As used herein, the term “coding region” refers to the nucleotide sequences that encode the amino acid sequences found in the nascent polypeptide as a result of translation of an mRNA molecule. The coding region is bounded in eukaryotes, on the 5′ side by the nucleotide triplet “ATG” that encodes the initiator methionine and on the 3′ side by one of the three triplets which specify stop codons (i.e., TAA, TAG, and TGA).

Where amino acid sequence is recited herein to refer to an amino acid sequence of a naturally occurring protein molecule, “amino acid sequence” and like terms, such as “polypeptide” or “protein,” are not meant to limit the amino acid sequence to the complete, native amino acid sequence associated with the recited protein molecule.

The term “wild-type” refers to a gene or gene product that has the characteristics of that gene or gene product when isolated from a naturally occurring source. A wild type gene is that which is most frequently observed in a population and is thus arbitrarily designed the “normal” or “wild-type” form of the gene.

In contrast, the terms “modified,” “mutant,” and “variant” refer to a gene or gene product that displays modifications in sequence and or functional properties (i.e., altered characteristics) when compared to the wild-type gene or gene product. It is noted that naturally occurring mutants can be isolated; these are identified by the fact that they have altered characteristics when compared to the wild-type gene or gene product.

The term “antibody” refers to polyclonal and monoclonal antibodies. Polyclonal antibodies which are formed in the animal as the result of an immunological reaction against a protein of interest or a fragment thereof, can then be readily isolated from the blood using well-known methods and purified by column chromatography, for example. Monoclonal antibodies can also be prepared using known methods (See, e.g., Winter and Milstein, Nature, 349, 293-299, 1991). As used herein, the term “antibody” encompasses recombinantly prepared, and modified antibodies and antigen-binding fragments thereof, such as chimeric antibodies, humanized antibodies, multifunctional antibodies, bispecific or oligo-specific antibodies, single-stranded antibodies and F(ab) or F(ab)₂ fragments. The term “reactive” in used in reference to an antibody indicates that the antibody is capable of binding an antigen of interest. For example, a β-catenin-reactive antibody is an antibody, which binds to β-catenin or to a fragment of β-catenin.

The term “dominant negative mutant” refers to molecules that lack wild type activity, but which effectively compete with wild type molecules for substrates, receptors, etc., and thereby inhibit the activity of the wild type molecule. In preferred embodiments, the terms “Lef1 dominant negative mutant” and “dnLef1” refer to a Lef1 mutant protein that competes with the wild type Lef1 protein for DNA substrates, but which fails to induce downstream effects.

The term “portion” when used in reference to a nucleotide sequence refers to fragments of that sequence, which range in size from 10 nucleotides to the entire nucleotide sequence minus one nucleotide.

The terms “mammals” and “mammalian” refer animals of the class mammalia, which nourish their young by fluid secreted from mammary glands of the mother, including human beings. The class “mammalian” includes placental animals, marsupial animals, and monotrematal animals.

The terms “patient” and “subject” refer to a human or other animal that is a candidate for receiving medical treatment.

As used herein, “proliferating” and “proliferation” refer to cells undergoing mitosis. Similarly, the “growth state” of a cell refers to the rate of proliferation of the cell and/or the state of differentiation of the cell. An “altered growth state” is a growth state characterized by an abnormal rate of proliferation (e.g., a cell exhibiting an increased or decreased rate of proliferation relative to a normal cell).

In some embodiments, the present invention provides methods and compositions for “retarding growth of cancer cell,” whereby different aspects of cancer cell growth are inhibited. As used herein, the term “retarding growth” indicates that the methods and compositions of the present invention inhibit proliferation of a cancer cell. In preferred embodiments, the “retarding growth” indicates that DNA replication (e.g., as measured by BrdU or tritiated thymidine incorporation) is at least 10% less than that observed in untreated or control-treated cancer cells. In particularly preferred embodiments, the term “retarding growth” indicates that DNA replication is at least 25% less than that observed in untreated or control-treated cancer cells. In still further preferred embodiments, the term “retarding growth” indicates that DNA replication is at least 50% (e.g., 75%, 90%, 95% or 99%) less than that observed in untreated or control-treated cancer cells.

In some preferred embodiments, the term “retarding growth” indicates that the methods and compositions of the present invention induce death (e.g., apoptosis) of a cancer cell. In preferred embodiments, the “retarding growth” indicates that apoptosis (e.g., as measured by FACS analysis and BrdU incorporation or any other suitable method) is at least 10% greater than that observed in untreated or control-treated cancer cells. In particularly preferred embodiments, the term “retarding growth” indicates that apoptosis is at least 25% greater than that observed in untreated or control-treated cancer cells. In still further preferred embodiments, the term “retarding growth” indicates that apoptosis is at least 50% (e.g., 75%, 90%, 95% or 99%) greater than that observed in untreated or control-treated cancer cells.

The term “control” refers to subjects or samples that provide a basis for comparison to experimental subjects or samples. For instance, the use of control subjects or samples permits determinations to be made regarding the efficacy of experimental procedures. In some embodiments, the term “control subject” refers to animals, which receive a mock treatment (e.g., vehicle alone).

As used herein, the terms “gene transfer” and “transfer of genetic information” refer to the process of moving a gene or genes from one place to another. In preferred embodiments of the present invention, the term “gene transfer” refers to the transfer of a polynucleotide to cells and/or tissues of an animal to achieve a therapeutic effect. In some embodiments, the polynucleotide may be in the form of a plasmid, a gene fragment or an oligonucleotide. In some embodiments, “gene transfer” is temporary or transient, in other embodiments “gene transfer” is sustained, and in still further embodiments, the gene transfer is long-lived, permanent or stable.

The terms “sample” and “specimen” in the present specification and claims are used in their broadest sense. On the one hand, they are meant to include a specimen or culture. On the other hand, they are meant to include both biological and environmental samples. In preferred embodiments, the term “sample” refers to biopsy material obtained from a subject's pancreas.

The term “carcinoma” refers to a malignant new growth made up of epithelial cells tending to infiltrate surrounding tissues and to give rise to metastases. Exemplary carcinomas include: “basal cell carcinoma”, which is an epithelial tumor of the skin that, while seldom metastasizing, has potentialities for local invasion and destruction; “squamous cell carcinoma”, which refers to carcinomas arising from squamous epithelium and having cuboid cells; “carcinosarcoma”, which include malignant tumors composed of carcinomatous and sarcomatous tissues; “adenocystic carcinoma”, carcinoma marked by cylinders or bands of hyaline or mucinous stroma separated or surrounded by nests or cords of small epithelial cells, occurring in the mammary and salivary glands, and mucous glands of the respiratory tract; “epidermoid carcinoma”, which refers to cancerous cells that tend to differentiate in the same way as those of the epidermis (e.g., tend to form prickle cells and undergo cornification); “nasopharyngeal carcinoma”, which refers to a malignant tumor arising in the epithelial lining of the space behind the nose; and “renal cell carcinoma”, which pertains to carcinoma of the renal parenchyma composed of tubular cells in varying arrangements. However, in preferred embodiments of the present invention, the term “carcinoma” refers to “adenocarcinoma,” which is a malignant tumor originating in glandular epithelium.

The terms “epithelia”, “epithelial” and “epithelium” refer to the cellular covering of internal and external body surfaces (cutaneous, mucous and serous), including the glands and other structures derived therefrom (e.g., corneal, esophegeal, epidermal, and hair follicle epithelial cells).

The term “Wnt” as used herein refers to a family of secreted signaling molecules that function during development. The term is used in reference to both the genes and their protein products. The first Wnt genes identified included wingless in Drosophila and int-1 in mice. The cell surface receptors for Wnt proteins are members of the Frizzled family of proteins containing seven transmembrane domains. Upon Wnt binding to Frizzled and the coreceptor LDL-receptor related protein (LRP), a cytoplasmic protein termed Dishevelled is activated. This leads to the inhibition of phosphorylation and degradation of β-Catenin, and β-Catenin mediated displacement of Groucho, resulting in transcription of Wnt target genes (e.g., c-myc). In contrast, in the absence of Wnt binding, Dishevelled is inactive, and β-Catenin is phosphorylated and degraded by a large protein complex comprising glycogen synthase kinase-3β (GSK-3β), adenomatous polyposis coli (APC) tumor suppressor protein, and axin. This results in continued Groucho co-repression of Wnt target genes.

Briefly, the term “Wnt signaling pathway”, “Wnt pathway” and “Wnt signal transduction pathway” are all used to refer to the chain of events normally mediated by Wnt, LRP, Frizzled, and β-catenin, among others, and resulting in a changes in gene expression and other phenotypic changes typical of Wnt activity. The Wnt pathway can be activated even in the absence of a Wnt protein, by activating a downstream component of the pathway. For example, some defective APC proteins activate the pathway in the absence of Wnt by permitting β-catenin accumulation in the cytosol and subsequent β-catenin migration into the nucleus.

The terms “Wnt antagonist” and “Wnt pathway inhibitor” refer to an agent that potentiates or recapitulates the bioactivity of GSK-3β, such as to repress transcription of Wnt-responsive genes. Preferred Wnt antagonists can be used to overcome LRP and/or Frizzled gain-of-function mutations (LRP and Frizzled antagonists, respectively). Other preferred Wnt antagonists can be used to overcome an inappropriate increase in Wnt signal transduction. A Wnt pathway inhibitor may be a small molecule, an antibody (including but not limited to: a single chain antibody, monoclonal antibody of IgG, IgM, IgA, IgD, or IgE isotype, or an antibody fragment comprising at least one pair of variable regions), an antisense nucleic acid, ribozyme, RNAi construct, or a mutant Wnt protein that can disrupt or inhibit Wnt signaling.

The non-steroidal anti-inflammatory compound sulindac (CAS Registry No. 38194-50-2; described in U.S. Pat. No. 3,654,349) is an exemplary Wnt pathway inhibitor. In particular, sulindac inhibits β-catenin/LCF-regulated transcription of target genes (Tutter et al., Genes Dev, 15:3342-3354, 2001; and Boon et al., Br J Cancer, 90:224-229, 2004).

The terms “Hedgehog” and “Hh” as used herein refer to a family of secreted signaling molecules that play important roles during development. The term is used in reference to both the genes and their protein products. In mammals, there are three hedgehog proteins (Sonic, Desert and Indian), whose responses are mediated by two transmembrane proteins, Patched and Smoothened. Specifically, upon Hedgehog binding to Patched, inhibition of Smoothened is removed, allowing Smoothened to relay a signal to a protein complex comprising a zinc finger-containing transcription factor Gli (homolog of Drosophila Cubitus interruptus). This leads to the cessation of Gli proteolysis and transcription of Hedgehog target genes. In contrast, in the absence of Hedgehog, Patched inhibits Smoothened signaling, resulting in proteolysis of Gli, and co-repression of Hedgehog target genes.

Briefly, the term “Hedgehog signaling pathway”, “Hedgehog pathway” and “Hedgehog signal transduction pathway” are all used to refer to the chain of events normally mediated by Hedgehog, Smoothened, Patched, and Gli, among others, and resulting in changes in gene expression and other phenotypic changes typical of hedgehog activity. The Hedgehog pathway can be activated even in the absence of a Hedgehog protein, by activating a downstream component of the pathway. For example, overexpression of Smoothened activates the pathway in the absence of Hedgehog.

The terms “Hedgehog antagonist” and “Hedgehog pathway inhibitor” refer to an agent that potentiates or recapitulates the bioactivity of Patched, such as to repress transcription of Hedgehog-responsive genes. Preferred Hedgehog antagonists can be used to overcome a Patched loss-of-function and/or a Smoothened gain-of-function mutation (the latter also being referred to as smoothened antagonists). Other preferred Hedgehog antagonists can be used to overcome an inappropriate increase in Hedgehog signal transduction. A Hedgehog pathway inhibitor may be a small molecule, an antibody (including but not limited to: a single chain antibody, monoclonal antibody of IgG, IgM, IgA, IgD, or IgE isotype, or an antibody fragment comprising at least one pair of variable regions), an antisense nucleic acid, ribozyme, RNAi construct, or a mutant Hedgehog protein that can disrupt or inhibit Hedgehog signaling.

The steroidal alkaloid cyclopamine (CAS Registry No. 4449-51-8; described in U.S. Pat. No. 2,789,977) is an exemplary Hedgehog pathway inhibitor. In particular, cyclopamine inhibits Hedgehog signal transduction by antagonizing Smoothened (Cooper et al., Science, 280:1603-1607, 1998; Incardona et al., Development, 125:3553-3562, 1998; and Taipale et al., Nature, 406:1005-1009, 2000).

The term “overexpression” as used in reference to gene expression levels means any level of gene expression in cells of a tissue that is higher than the normal level of expression for that tissue. The normal level of expression for a tissue is assessed by measuring gene expression in a healthy portion of the tissue of interest.

The term “prodrug” is intended to encompass compounds that, under physiological conditions, are converted into the therapeutically active agents of the present invention. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “antisense molecule” refers to polynucleotides and oligonucleotides capable of binding to an mRNA molecule. In particular, an antisense molecule is a DNA or RNA sequence complementary to an mRNA sequence of interest. In preferred embodiments, the term β-catenin antisense molecule refers to a single-stranded DNA or RNA sequence that binds to at least a portion of a β-catenin mRNA molecule to form a duplex which then blocks further transcription and/or translation.

The term “RNA interference” or “RNAi” refers to the silencing or decreasing of gene expression by iRNA or siRNAs. It is the process of sequence-specific, post-transcriptional gene silencing in animals and plants, initiated by iRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “interfering RNA (iRNA)” refers to a double stranded RNA molecule that mediates RNA interference (RNAi). At least one strand of the duplex or double-stranded region of an iRNA is substantially homologous to or substantially complementary to a target RNA molecule. The strand complementary to a target RNA molecule is the “antisense strand;” the strand homologous to the target RNA molecule is the “sense strand,” and is also complementary to the iRNA antisense strand. iRNAs may also contain additional sequences; non-limiting examples of such sequences include linking sequences, or loops, as well as stem and other folded structures.

The iRNA can serve as a source of siRNA. siRNAs generally comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of an iRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

As used herein, the term “loop sequence” refers to a nucleic acid sequence located between two nucleic sequences that are complementary to each other and which forms a loops when the complementary nucleic acid sequences hybridize to one another.

### GENERAL DESCRIPTION OF THE INVENTION

Pancreatic adenocarcinoma is a devastating disease marked by rapid progression and metastatic complications. During development of the present invention, Wnt signaling, a pathway that interacts with the Hedgehog pathway during embryogenesis was found to be active in human pancreatic adenocarcinoma. Inhibition of Wnt signaling blocks proliferation of adenocarcinoma cells, and arrests tumor growth in xenotransplantation experiments. Moreover, Hedgehog signaling, previously implicated in initiation of pancreatic tumor formation was also found to regulate Wnt pathway activity. These studies indicate that there is a hierarchical relationship among embryonic signaling pathways in pancreatic carcinogenesis. Thus, the present invention provides methods and compositions comprising the use of Wnt and Hedgehog pathway inhibitors for reducing proliferation of pancreatic adenocarcinoma cells.

### DETAILED DESCRIPTION OF THE INVENTION

Interactions between embryonic signaling pathways ensure proper organ formation during development. Increasing evidence suggests that these pathways remain active in a subset of cells within adult organs and that deregulation of their activity contributes to the formation and maintenance of certain tumors (Pasca di Magliano and Hebrok, Nature Rev Cancer, 3:903-911, 2003). Wnt signaling is one of such pathways involved in organogenesis and tumor formation within the gastrointestinal tract (Heller et al., Dev Dyn, 225:260-270, 2002; and Lustig and Behrens, J Cancer Res Clin Oncol, 129:199-221, 2003). In the canonical branch of the Wnt pathway, soluble Wnt ligands form a complex with one of several frizzled receptors and Lrp5/Lrp6 co-receptors. This interaction triggers a cascade of events that result in the stabilization of β-catenin, an intracellular protein that first accumulates in the cytoplasm and subsequently translocates to the nucleus. Binding of β-catenin to transcription factors of the Tcf-Lef family activates the transcription of Wnt target genes (Hecht and Kemler, EMBO Rep, 1:24-28, 2003) and deregulation of β-catenin activation has been associated with several gastrointestinal tumors (Lustig and Behrens, supra, 2003). Recently the nuclear localization of β-catenin in a subset of pancreatic adenocarcinomas was found to correlate with the severity of the diagnosis (Watanabe et al., Pancreas, 26:326-333, 2003). However, prior to development of the present invention functional studies addressing the contribution of the Wnt signaling pathway to the generation and maintenance of pancreatic adenocarcinoma were lacking. As described herein, the present invention provides methods and compositions comprising Wnt and Hedgehog pathway inhibitors for reducing proliferation of pancreatic adenocarcinoma cells.

### I. Wnt Signaling in Pancreatic Cancer

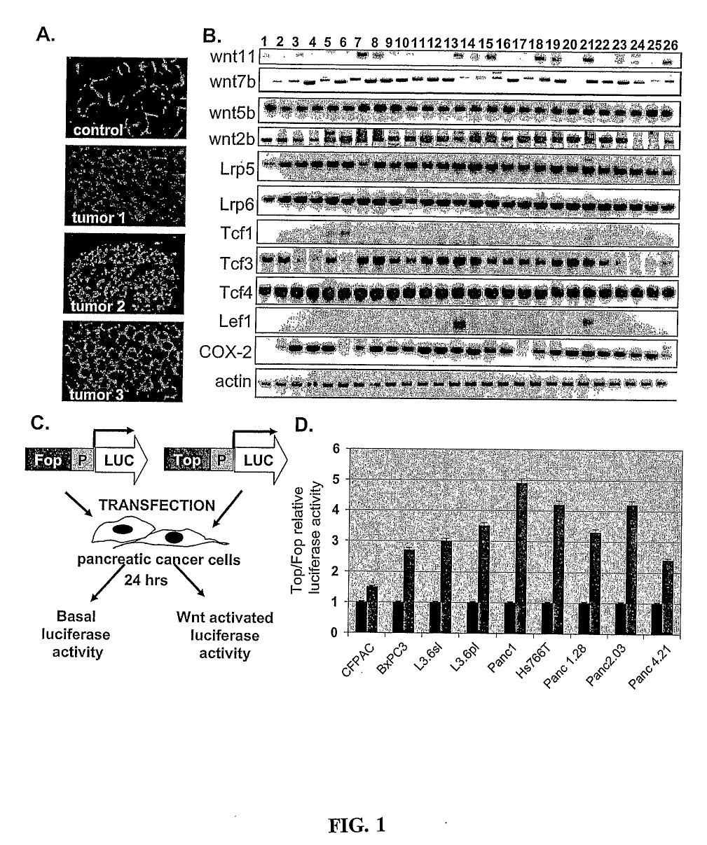

As described herein, activation of Wnt signaling is a common trait in pancreatic adenocarcinoma. Analysis of a panel of primary human tumor tissues revealed nuclear or cytoplasmic accumulation of β-catenin in 43% percent of the samples (6/14 samples, representatives of which are shown in FIG. 1A). Furthermore, expression of Wnt signaling components, including Wnt ligands (Wnt1 l, Wnt7b, Wnt5b, and Wnt2b), the Wnt ligand co-receptors Lrp5 and Lrp6, and the transcription factors Tcf3 and Tcf4, was observed in all of 26 human pancreatic cancer cell lines derived from either primary or metastatic pancreatic adenocarcinoma (See, FIG. 1B). In contrast, expression of Tcf1 and Lef1, transcription factors of the canonical signaling arm in other tissues are expressed in only a subset of the cell lines. Measurement of luciferase expression controlled by concatemers of ‘TCF optimal sites’ (Top) upstream of a minimal thymidine kinase (TK) promoter element allows quantitative evaluation of Wnt signaling activity. The use of concatemers of TCF ‘far from optimal sites’ (Fop) upstream of the TK-promoter permits the measurement of Wnt-independent (basal) activity of the reporter construct. Using this reporter system shown in the schematic of FIG. 1C (van de Wetering et al., Cell, 88:789-799, 1997), Wnt-signaling was detected (activation of the Top-Flash reporter) in nine different adenocarcinoma cell lines (See, FIG. 1D), indicating that active Wnt signaling is a common marker of human pancreatic adenocarcinoma cells. Furthermore, a majority of the cell lines also were found to express COX-2, a transcriptional target gene of Wnt signaling, which is often elevated in pancreatic adenocarcinoma and other human tumors (Araki et al., Cancer Res, 63:728-734, 2003; Molina et al., Cancer Res, 59:4356-4362, 1999; and Eberhart et al., Gastroenterology, 107:1183-1188, 1994). A subset of the pancreatic adenocarcinoma cell lines was chosen for further analysis including well-established lines (CFPAC and BxPC3), lines with increased metastatic potential (L3.6sl), and lines closely resembling the primary tumor phenotype (Panc4.21). All four lines were found to express significant levels of non-phosphorylated β-catenin, another reliable marker of active Wnt/β-catenin signaling.

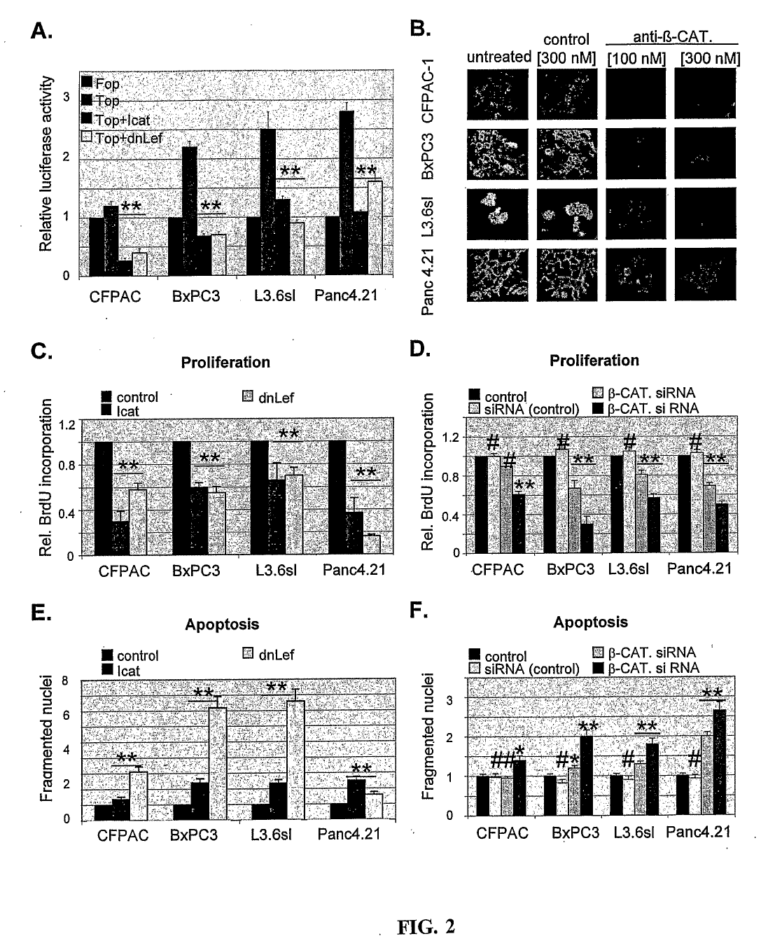

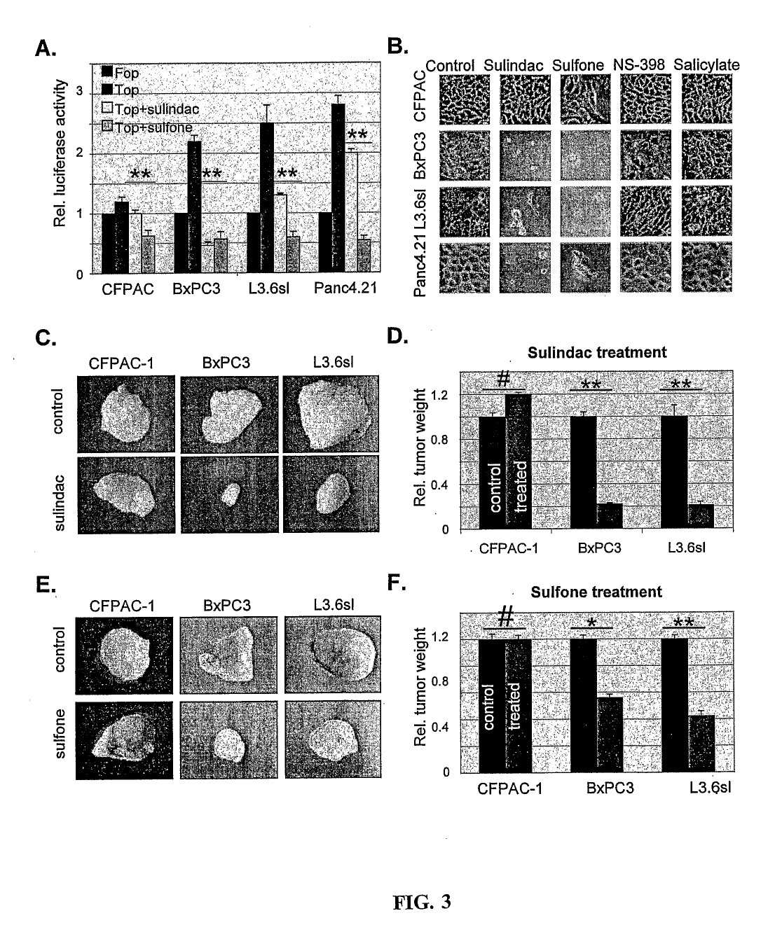

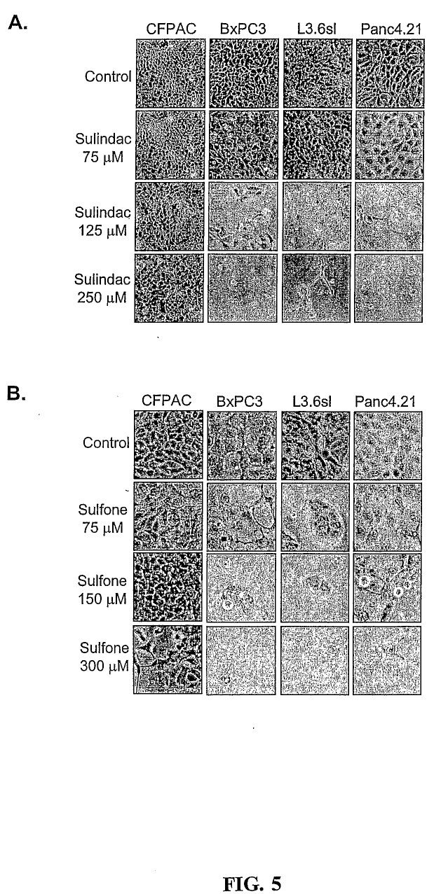

An increasing number of studies describe the potential of small chemical compounds to act as regulators of intercellular signaling processes. Sulindac, a member of the family of non-steroidal anti-inflammatory drugs (NSAIDs), is known to induce apoptosis in tumor cells via inhibition of Wnt signaling (Shoff and Rigas, J Exp Med, 190:445-450, 1999). To test whether sulindac can block Wnt signaling in pancreatic adenocarcinoma cells four cell lines (CFPAC-1, BxPC3, L3.6sl, and Panc4.21) were transfected with the Top/Fop-Flash reporter constructs. Sulindac treatment suppressed Top-Flash reporter activation in all cell lines tested, however, with varying efficiency as shown in FIG. 3A. Similarly, co-transfection with endogenous Wnt inhibitors ICAT (Tago et al., Genes Develop, 14:1741-1749, 2000), or dominant negative Lef1 (dn-Lef-1), also resulted in reduction of Top-Flash activity in pancreatic adenocarcinoma cells, as shown in FIG. 2A. Importantly, sulindac treatment induced apoptosis (two to four-fold) and impaired cell proliferation (between 50 and 99%) in the BxPC3, L3.6sl, and Panc4.21 cell lines in a concentration dependent manner, while CFPAC-1 cells were only marginally affected (See, FIG. 3B and FIG. 5A). As shown in Table 1, further analysis of eight additional lines revealed measurable reduction in cell proliferation in 54% of the lines tested (7/13).

TABLE 1
Sensitivity of Pancreatic Adenocarcinoma Cells to Wnt Pathway
Inhibition

| Cell Line | Sulindac Sensitivity |
|---|---|
| Panc1 | — |
| Hs766T | — |
| CFPAC-1 | — |
| BxPC3 | Positive |
| AsPC1 | — |
| L3.6sl | Positive |
| L3.6pl | Positive |
| Panc1.28 | — |
| Panc2.03 | Positive |
| Panc3.27 | Positive |
| Panc4.21 | Positive |
| Panc6.03 | Positive |
| Panc10.05 | — |

Sulindac is a pro-drug that is metabolized into a sulfide and a sulfone. Both metabolites have been shown to induce caspase- and proteasome-dependent β-catenin degradation in colon cancer cells (Rice et al., Mol Cancer Ther, 2:885-892, 2003). However, in contrast to sulindac sulfide, the sulfone does not block cycloxygenases. Sulindac sulfone is thought to stimulate protein kinase G (PKG) activity via inhibition of cyclic nucleotide phosphodiesterase (Piazza et al., Cancer Res, 57:2452-2459, 1997; and Thompson et al., Cancer Res, 60:3338-3342, 2000). PKG in turn is thought to phosphorylate residues located in the C-terminal portion of β-catenin, thereby marking it for degradation (Li et al., Nat Genet, 23:67-70, 1999). Sulindac sulfone treatment blocked Wnt signaling and cell survival in cultured pancreatic cancer cell lines as shown in FIGS. 3B and 5B. To confirm that the growth inhibition observed in sulindac-treated cells was not mediated through inhibition of COX-2 activity cell growth was examined in the presence of two different COX-2 inhibitors. Neither NS-398 (blocks COX-2 activity) nor salicylate (blocks COX-2 transcription) showed any measurable effect on pancreatic cancer cell growth or survival (See, FIG. 3B). Taken together, these data indicate that inhibition of Wnt, as opposed to inhibition of COX-2, contributes to attenuation of cell proliferation in pancreatic adenocarcinoma cells. Further evidence of sulindac-mediated inhibition of Wnt signaling is provided by the finding that the expression of downstream mediators of Wnt signaling, including Tcf1 and Tcf4, is reduced in sulindac-treated pancreatic cancer cells (See, FIG. 2E).

Sulindac metabolites are known to interfere with other signaling pathways, including the ras pathway (Rice et al., Cancer Res, 61:1541-1547, 2001; and Herrmann et al., Oncogene, 17:1769-1776, 1998). To confirm that inhibition of Wnt signaling is sufficient to block cell proliferation, BrdU incorporation was measured in cells transiently transfected with the endogenous Wnt inhibitor Icat (Tago et al., Genes Devel, 14:1741-1749, 2000) as shown in FIGS. 2C and 2E. The presence of an IRES sequence driving expression of eGFP in the lcat-construct allowed sorting and comparison of lcat-GFP negative control and lcat-GFP positive cells by FACS. Cell proliferation in all pancreatic adenocarcinoma cell lines tested was significantly reduced, indicating that active Wnt signaling is essential for pancreatic tumor cell growth. Previous studies have shown effective reduction of β-catenin and Wnt signaling in colon cancer cell lines upon treatment with siRNA's directed against β-catenin (Verma et al., Clin Cancer Res, 9:1291-1300, 2003). Using a similar strategy, a significant reduction in β-catenin protein levels was observed in CFPAC-1, BxPC3, L3.6sl and Panc4.21 pancreatic cancer cells. More importantly, anti-β-catenin siRNA treatment of pancreatic adenocarcinoma cell lines efficiently reduced cell proliferation and induced apoptosis in a concentration dependent manner (See, FIGS. 2D and 2F). In summary, results from several independent experimental strategies described herein, indicate that constitutive Wnt signaling is required for growth of pancreatic cancer cells.

While effects of sulindac and sulindac sulfone that are independent of their Wnt-inhibitory activity cannot be unequivocally excluded, it is important to note that the level of cancer cell growth inhibition mediated by these compounds is in direct proportion to their respective ability to block Wnt signaling. For example, sulindac sulfone is generally more effective than sulindac in blocking Wnt signaling (FIG. 3A) and even diminishes growth of CFPAC cells that are only marginally affected by sulindac treatment (See, FIGS. 3B; 5A and 5B). Furthermore, sulindac does not act as a general cytotoxic agent as it only blocks cell proliferation in a subset of human adenocarcinoma cell lines (See, Table 1).

Formation of solid tumors requires anchorage-independent growth within the three dimensional matrix of the surrounding tissue. To test whether sulindac inhibits tumor growth after xenotransplantation, two highly responsive cell lines (BxPC3 and L3.6sl) and a marginally responsive cell line (CFPAC-1), were injected subcutaneously into immunocompromised nude mice. Once tumors were palpable, mice were treated with either sulindac or control vehicle for a total of five days. No reduction in control or sulindac-treated CFPAC-1 tumor volume was detected at the end of the treatment period (See, FIGS. 3C and 3D). In contrast, sulindac treated BxPC3 and L3.6sl tumors displayed an 80% reduction in volume when compared to untreated samples.

To determine whether tumor reduction can be achieved independent of COX inhibition, mice carrying palpable tumors were treated with the Wnt inhibitor sulindac sulfone, which lacks the ability to inhibit COX activity (Thompson et al., Cancer Res, 60:3338-3342, 2000; and Li et al., Cancer Biol Ther, 1:621-625, 2002). The limited solubility of sulindac sulfone required oral application of the compound as opposed to subcutaneous application of sulindac. Albeit by a different method of application, sulindac sulfone-treatment reduced the mass of responsive L3.6sl and BxPC3 tumors by 50% and 60%, respectively, while the size of CFPAC-1 tumors remained largely unaffected (See, FIGS. 3E and 3F). The more potent effects observed with sulindac compared to sulindac sulfone are contemplated to be due to intrinsic properties of the different inhibitors, including bioavailability and halftime of the active compounds. Alternatively, it is possible that sulindac sulfide has additional activities beyond the inhibition of Wnt signaling and COX activities.

### II. Hedgehog Signaling in Pancreatic Cancer

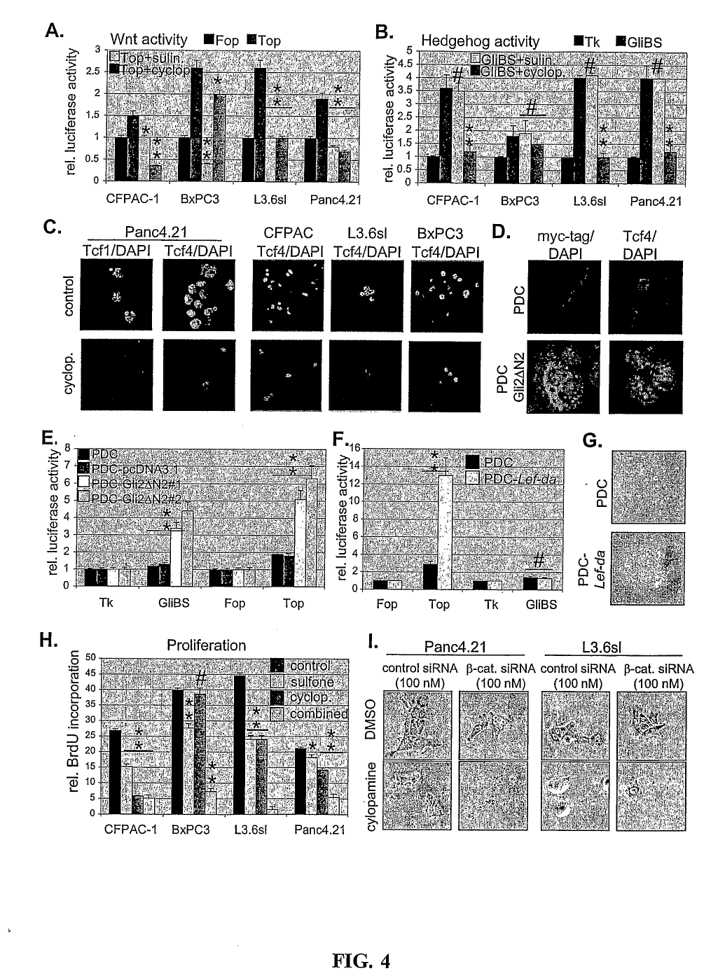

Inhibition of Hedgehog signaling with the cholesterol-derivative cyclopamine has previously been shown to arrest proliferation of digestive tract tumors (Berman et al., Nature, 425:846-851; and Thayer et al., Nature, 425:851-856, 2003). This is somewhat surprising given that cyclopamine treatment had been shown to promote ectopic pancreatic tissue development in the embryonic chick (Kim and Melton, Proc Natl Acad Sci USA, 95:13036-13041, 1998). In addition, Hedgehog and Wnt signaling are known to interact during mammalian organ development (Pinson et al., Nature, 407:535-538, 2000; and Parr and McMahon, Nature, 374, 350-353, 1995). Thus, it was necessary to elucidate the nature of the Hedgehog/Wnt relationship in pancreatic cancer cells. This was accomplished during development of the present invention by transfection of CFPAC-1, BxPC3, L3.6sl, and Panc4.21 cell lines with either a Top-Flash Wnt reporter construct or a GliBS-luc Hedgehog reporter construct (See, FIGS. 4A and 4B). Transfected cells were either treated with cyclopamine, a hedgehog inhibitor, or sulindac. Cyclopamine inhibits the activity of the Hedgehog reporter construct in pancreatic cancer cells (FIG. 4B). Treatment with cyclopamine also strongly reduced the activity of the Wnt reporter (FIG. 4A) in select cell lines (CFPAC-1, L3.6sl, and Panc4.21). In addition, cyclopamine results in a comparable, marginal reduction of both Hedgehog (17%) as well as Wnt signaling (˜23%) in BxPC3 cells (See, FIGS. 4A and 4B), indicating that reduction of Hedgehog signaling impairs Wnt activity. In contrast, treatment with sulindac showed an inhibitory effect on the Top-Flash Wnt reporter (FIG. 4A), but had no affect on activation of the GliBS-luc Hedgehog reporter in any of the cell lines tested (FIG. 4B). Thus as described herein for the first time, Hedgehog signaling regulates Wnt activity in pancreatic adenocarcinoma cells. These findings also underscore the fact that sulindac-mediated effects on cell proliferation and apoptosis are not caused by inhibition of Hedgehog activity. Nonetheless, knowledge of the mechanism(s) is not necessary in order to make and use the present invention.

### III. Wnt and Hedgehog Signaling in Pancreatic Cancer

During development of the present invention, the affect of cyclopamine treatment (Hedgehog inhibition) on expression of Tcf genes (known mediators of Wnt signaling and target genes of the pathway) was tested. In general, Tcf1 RNA expression is low in most pancreatic adenocarcinoma cell lines (FIG. 1B), although expression of the Tcf1 protein was detected by immunohistochemistry in Panc4.21 cells. Cyclopamine was found to reduce expression of the Tcf1 protein in these cells (FIG. 4C). Additionally, expression of Tcf4, a mediator of Wnt signaling found in all pancreatic adenocarcinoma cell lines (FIG. 1B), was efficiently abolished by cyclopamine treatment (FIG. 4C). While analysis of pancreatic cancer cell lines has provided some insights into how signaling pathways are required for tumor growth and maintenance, studies of transformed cells cannot address the signaling event that induce tumorigenesis in the pancreas.

Previously, the deregulation of Hedgehog signaling was found to be sufficient to initiate tumor formation in mice (Thayer et al., Nature, 425:851-856, 2003). Cells isolated from pancreatic ducts (pancreatic ductal cells or PDCs, See, Schreiber et al., Gastroenterology, 127:250-260, 2004) were used to determine whether deregulation of Hedgehog signaling also controls Wnt activity in non-transformed cells. Ectopic activation of Hedgehog activity via expression of a dominant-active version of Gli2 (Gli2ΔN2, See, Sheng et al., Cancer Res, 62:5308-5316, 2002) was found to induce Tcf4 expression (FIG. 4D). Furthermore, activation of Hedgehog via ectopic expression of either Gli2ΔN2 or a dominant-active form of Smoothened (SmoA1) was found to significantly upregulate GliBs-Luciferase and Top-Flash activity in untransformed PDCs (FIG. 4E). Thus, Hedgehog regulates Wnt activity in untransformed pancreatic duct cells, as well as pancreatic cancer cells, at least in part via control of Tcf expression.

PDCs were transfected with an expression vector coding for a dominant-negative form of Lef1 (Lef1-da) followed by an IRES-eGFP sequence, to test whether Wnt signaling regulates Hedgehog activity. Target gene expression in pools of transfected cells was verified by western blot and by FACS scan for eGFP. Top-Flash reporter assays confirmed an approximately 4-fold increase in Wnt activity in PDC-Lef1-da transfected cells. However, ectopic activation of Wnt signaling did not induce luciferase activity of the Hedgehog responsive GliBS reporter (FIG. 4F). Although PDC-Lef1-da cells have acquired the ability for anchorage-independent growth allowing the formation of colonies when grown in soft agar (FIG. 4G), subcutaneous transplantation of PDC-Lef1-da cells in nude mice did not result in formation of tumors. Thus, as described herein ectopic activation of Wnt signaling itself is not sufficient to induce pancreatic tumors.

### IV. Inhibition of Wnt and Hedgehog Signaling in Pancreatic Cancer

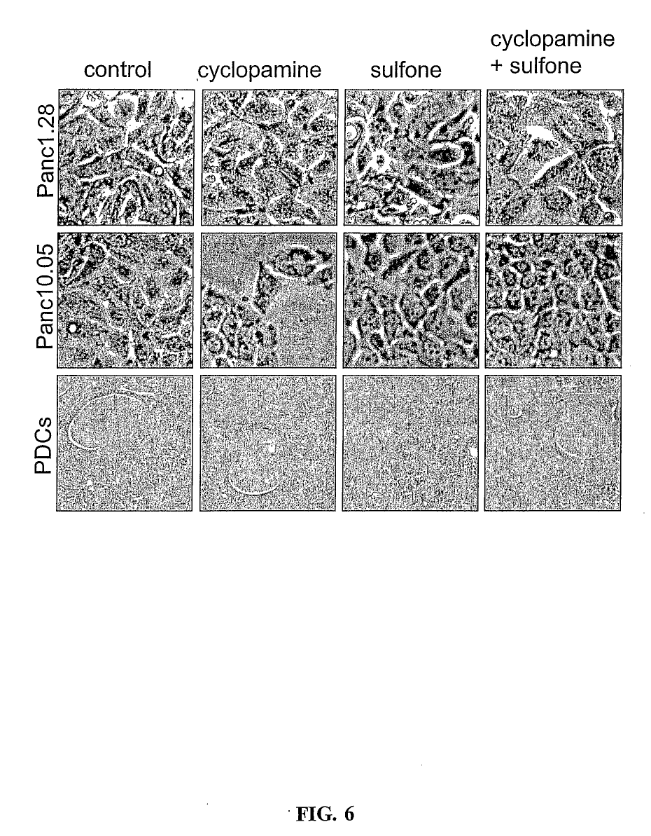

A treatment protocol comprising simultaneously contacting pancreatic adenocarcinoma cell lines with a combination of sulindac sulfone and cyclopamine (both individual antagonists were used at half the concentration of previous experiments) was designed to determine whether simultaneous Wnt and Hedgehog inhibition results in increased loss of cell proliferation. As shown herein for the first time (FIG. 4H), the combined treatment resulted in a significantly greater reduction of cancer cell proliferation in multiple pancreatic adenocarcinoma cell lines (BxPC3, L3.6sl, and Panc4.21). Similar results were obtained when sulindac sulfone was replaced by anti-βcatenin siRNA (FIG. 4I), a more specific inhibitor of Wnt signal transduction. In particular, cancer cells treated simultaneously with anti-β-catenin siRNA and cyclopamine ceased proliferating and appeared aplastic. Thus, the present invention provides novel methods comprising the co-administration of Hedgehog and Wnt pathway inhibitors, for achieving a greater anti-proliferative affect than that obtained from the use of either inhibitor alone. The synergistic anti-proliferative affect obtained by using both Hedgehog and Wnt pathway inhibitors is particularly promising for cancer chemotherapy, since the simultaneous inhibition of both pathways does not appear to be indiscriminately cytotoxic. Evidence of the selective affect of Hedgehog and Wnt pathway inhibitors on growth of certain types of pancreatic cancer cells is provided herein. Briefly, the images provided in FIG. 6 indicate that the simultaneous inhibition of both pathways does not affect survival of pancreatic cancer cell lines that are insensitive to cyclopamine or sulindac sulfone treatment, and does not affect the survival of untransformed pancreatic duct cells.

In contrast, recent studies have demonstrated mutually inhibitory interactions between Hedgehog and Wnt signaling cascades in colonic epithelial cell differentiation (van den Brink et al., Nat Genet, 36:277-282, 2004). Deregulated Wnt signaling, a common feature in human colon cancer, blocks Hedgehog signaling in these cells. Thus, the relationship between the Wnt and Hedgehog pathways in colon cancer is clearly different from and not predictive of the hierarchical relationship observed in certain human pancreatic adenocarcinoma cells. It follows that distinct interactions occur between embryonic signaling pathways within developing gastrointestinal tract tissues and tumors. Notably, Hedgehog and Wnt signaling appear to be critical for both the development and survival of pancreatic adenocarcinoma cells. The finding that co-treatment with Wnt and Hedgehog inhibitors reduces pancreatic cancer cell proliferation more potently than treatment with individual antagonists provides improved treatment options for patients with this generally fatal disease. Thus, the use of cyclopamine and sulindac or anti-β-catenin siRNA or other inhibitors of the Hedgehog and Wnt pathways (Frank-Kamenestsky et al., J Biol, 1:10, 2002; and Lepourcelet et al., Cancer Cell, 5:91-102, 2004) is contemplated to provide a greater therapeutic benefit than that obtained from the use of either inhibitor alone.

### V. Wnt Pathway Inhibitors

Wnt pathway inhibitors suitable for use with the methods of the present invention include but are not limited to small molecules (e.g., molecular weight less than 2 kDa), antibodies, recombinant proteins, and antisense RNAs (e.g. dsRNA molecules and siRNA vector constructs, etc.). In some embodiments of the present invention Wnt pathway inhibitors are provided in the form of antagonists, while in other embodiments the Wnt pathway inhibitors are provided in the form of agonists. For instance, suitable Wnt pathway antagonists include but are not limited to compounds that antagonize one or more of LRP, Frizzled, Wnt, Dishevelled, β-catenin (e.g., β-catenin siRNA), Lef and Tcf. Suitable Wnt pathway agonists include but are not limited to compounds that agonize one or more of GSK-3β, APC, Dickkopf, Axin1, and Axin2 (also called Conductin, or Axil). Regardless of the mechanism of action, Wnt pathway inhibitors that find use with the present invention include compounds suitable for maintaining repression of Wnt-responsive genes. The non-steroidal anti-inflammatory drugs sulindac sulfide and sulindac sulfone are disclosed herein, simply as exemplary Wnt pathway inhibitors. Additional substituted indenyl acetic acids, and derivatives of sulindac are contemplated to be appropriate alternatives to sulindac for use in the methods and compositions of the present invention. Other suitable Wnt antagonists include but are not limited to Wnt-inhibitory factor-1 (WIF-1), and Frizzled-related protein (FRP). Table 2 provides a listing of Wnt pathway components and their corresponding GENBANK accession numbers (nucleotide and amino acid sequences herein incorporated by reference), which can be used for the rational design of Wnt pathway inhibitors.

TABLE 2
Components of the Wnt Signaling Pathway

| Wnt Component | GENBANK Accession No. |
|---|---|
| WNT1 | NM_005430 |
| WNT2 | NM_00339 |
| WNT2B/13 | NM_024494 |
| WNT3 | NM_030753 |
| WNT3A | NM_033131 |
| WNT4 | NM_030761 |
| WNT5A | NM_003392 |
| WNT5B | NM_032642 |
| WNT6 | NM_006522 |
| WNT7A | NM_004625 |
| WNT7B | NM_058238 |
| WNT8A | NM_058244 |
| WNT8B | NM_003393 |
| WNT9A (WNT14) | NM_003395 |
| WNT9B (WNT15) | NM_003396 |
| WNT10A | NM_025216 |
| WNT10B | NM_003394 |
| WNT11 | NM_004626 |
| WNT16 | NM_057168 |
| FRIZZLED | NM_003505 |
| FRIZZLED2 | NM_001466.2 |
| FRIZZLED3 | NM_017412 |
| FRIZZLED4 | NM_012193 |
| FRIZZLED5 | NM_003468 |
| FRIZZLED6 | NM_003506 |
| FRIZZLED7 | NM_003507 |
| FRIZZLED8 | NM_031866 |
| FRIZZLED9 | NM_003508 |
| FRIZZLED10 | NM_007197 |
| LRP5 | NM_002335 |
| LRP5 | NM_002336 |
| TCF1 (TCF7)* | NM_003202, NM_201632, NM_201633 |
| TCF3 (TCF7L1) | NM_031283 |
| TCF4 (TCF7L2) | NM_030756 |
| LEF1 | NM_016269 |
| β-CATENIN | NM_001904 |
| PLAKOGLOBIN* | NM_002230, NM_021991 |
| DISSHEVELLED1 (DVL1)* | NM_004421, NM_181870, NM_182779 |
| DISSHEVELLED2 (DVL2) | NM_004422 |
| DISSHEVELLED3 (DVL3) | NM_004423 |
| DISSHEVELLED4 (Dvl1L1) | HSU46462 |

*Multiple GENBANK Accession numbers correspond to alternative transcripts.

### VI. Hedgehog Pathway Inhibitors

Hedgehog pathway inhibitors suitable for use with the methods of the present invention include but are not limited to small molecules (e.g., molecular weight less than 2 kDa), antibodies, recombinant proteins, and antisense RNAs (e.g., dsRNA molecules and siRNA molecules). In some embodiments of the present invention Hedgehog pathway inhibitors are provided in the form of antagonists, while in other embodiments the Hedgehog pathway inhibitors are provided in the form of agonists. For instance, suitable Hedgehog pathway antagonists include but are not limited to compounds that antagonize one or more of Smoothened, Sonic Hedgehog, Desert Hedgehog, Indian Hedgehog, Gli1 and Gli2 (e.g., Gli siRNA). Suitable Hedgehog pathway agonists include but are not limited to compounds that agonize one or more of Patched (e.g. maintain Smoothened inhibition by for instance blocking Hedgehog binding) and suppressor of fused (e.g., prevent accumulation and translocation of intact Gli into the nucleus). Regardless of the mechanism of action, Hedgehog pathway inhibitors that find use with the present invention include compounds such as the anti-hedgehog antibody 5E1, Cur61414, and forskolin (See, e.g., Frank-Kamenestsky et al., J Biol, 1:10, 2002, published online) that are suitable for maintaining repression of Hedgehog-responsive genes. Cyclopamine (11-deoxojervine) derived from Veratrum californicum, is disclosed herein simply as an exemplary Hedgehog pathway inhibitor. Additional veratrum alkaloids, and derivatives of cyclopamine are contemplated to be appropriate alternatives to cyclopamine for use in the methods and compositions of the present invention. Other suitable Hedgehog antagonists include but are not limited to the Hedgehog-interacting protein (HIP), and compositions disclosed in U.S. Patent Application Publication US 2004/0110663A1 (herein incorporated by reference in its entirety). Table 3 provides a partial listing of Hedgehog pathway components and their corresponding GENBANK accession numbers (nucleotide and amino acid sequences herein incorporated by reference), which can be used for the rational design of Hedgehog pathway inhibitors.

TABLE 3
Components of the Hedgehog Signaling Pathway

| Hedgehog Component | GENBANK Accession No. |
|---|---|
| SMOOTHENED | NM_005631 |
| SONIC HEDGEHOG | NM_000193 |
| INDIAN HEDGEHOG | NM_002181 |
| DESERT HEDGEHOG | NM_021044 |
| GLI1 | NM_005269 |
| GLI2* | NM_030379, NM_030380, NM_030381, |
| | NM_005270 |

*Multiple GENBANK Accession numbers correspond to alternative transcripts.

### VII. Inhibition of Wnt and Hedgehog Signaling via RNAi

RNAi refers to the introduction of homologous double stranded RNA (dsRNA) to target a specific gene product, resulting in post-transcriptional silencing of that gene. This phenomena was first reported in Caenorhabditis elegans by Guo and Kemphues (Cell, 81:611-620, 1995) and subsequently Fire an colleagues Nature 391: 806-811, 1998) discovered that it is the presence of dsRNA, formed from the annealing of sense and antisense strands present in the in vitro RNA preps, that is responsible for producing the interfering activity.

The present invention contemplates the use of RNA interference (RNAi) to down-regulate the expression of Wnt and Hedgehog genes dysregulated in pancreatic adenocarcinoma cells, thus inhibiting proliferation and/or inducing apoptosis. In both plants and animals, RNAi is mediated by RNA-induced silencing complex (RISC), a sequence-specific, multi-component nuclease that destroys messenger RNAs homologous to the silencing trigger. RISC is known to contain short RNAs (approximately 22 nucleotides) derived from the double-stranded RNA trigger, although the protein components of this activity are unknown. However, the 22-nucleotide RNA sequences are homologous to the target gene that is being suppressed. Thus, the 22-nucleotide sequences appear to serve as guide sequences to instruct a multi-component nuclease, RISC, to destroy the specific mRNAs. More recently, Carthew has reported (Curr Opin Cell Biol, 13:244-248, 2001) that eukaryotes silence gene expression in the presence of dsRNA homologous to the silenced gene. Biochemical reactions that recapitulate this phenomenon generate RNA fragments of 21 to 23 nucleotides from the double-stranded RNA. These stably associate with an RNA endonuclease, and probably serve as a discriminator to select mRNAs. Once selected, mRNAs are cleaved at sites 21 to 23 nucleotides apart.

In preferred embodiments, the dsRNA used to initiate RNAi, may be isolated from native source or produced by known means (e.g., transcribed from DNA). RNA is synthesized either in vivo or in vitro. In some embodiments, endogenous RNA polymerase of the cell may mediate transcription in vivo, or cloned RNA polymerase can be used for transcription in vivo or in vitro. In other embodiments, the RNA is provided by transcription from a transgene in vivo or an expression construct. In some embodiments, the RNA strands are polyadenylated; in other embodiments, the RNA strands are capable of being translated into a polypeptide by a cell's translational apparatus. In still other embodiments, the RNA is chemically or enzymatically synthesized by manual or automated reactions. In further embodiments, the RNA is synthesized through use of a cellular RNA polymerase or a bacteriophage RNA polymerase (e.g., T3, T7, SP6). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. In some embodiments, the RNA is dried for storage or dissolved in an aqueous solution. In other embodiments, the solution contains buffers or salts to promote annealing, and/or stabilization of the duplex strands.

In some embodiments, the dsRNA is transcribed from the vectors as two separate stands. In other embodiments, the two strands of DNA used to form the dsRNA may belong to the same or two different duplexes in which they each form with a DNA strand of at least partially complementary sequence. When the dsRNA is thus produced, the DNA sequence to be transcribed is flanked by two promoters, one controlling the transcription of one of the strands, and the other that of the complementary strand. These two promoters may be identical or different. In some embodiments, a DNA duplex provided at each end with a promoter sequence can directly generate RNAs of defined length, which are then joined in pairs to form a dsRNA (See, e.g., U.S. Pat. No. 5,795,715, incorporated herein by reference). RNA duplex formation may be initiated either inside or outside the cell.

Inhibition is sequence-specific in that nucleotide sequences corresponding to the duplex region of the RNA are targeted for genetic inhibition. RNA molecules containing a nucleotide sequence identical to a portion of the target gene are preferred for inhibition. RNA sequences with insertions, deletions, and single point mutations relative to the target sequence have also been found to be effective for inhibition. Thus, sequence identity may optimized by sequence comparison and alignment algorithms known in the art and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript. The length of the identical nucleotide sequences may be at least 25, 50, 100, 200, 300 or 400 bases.

There is no upper limit on the length of the dsRNA that can be used. For example, the dsRNA can range from about 21 base pairs (bp) of the gene to the full length of the gene or more. In one embodiment, the dsRNA used in the methods of the present invention is about 1000 bp in length. In another embodiment, the dsRNA is about 500 bp in length. In yet another embodiment, the dsRNA is about 22 bp in length.

In some preferred embodiments, the sequences that mediate RNAi are from about 21 to about 23 nucleotides. That is, the isolated RNAs of the present invention mediate degradation of the target RNA (e.g., β-catenin). The double stranded RNA of the present invention need only be sufficiently similar to natural RNA that it has the ability to mediate RNAi for the target RNA. In one embodiment, the present invention relates to RNA molecules of varying lengths that direct cleavage of specific mRNA to which their sequence corresponds. It is not necessary that there be perfect correspondence of the sequences, but the correspondence must be sufficient to enable the RNA to direct RNAi cleavage of the target mRNA. In a particular embodiment, the RNA molecules of the present invention comprise a 3′ hydroxyl group. In some embodiments, the amount of target RNA (mRNA) is reduced in the cells of the target tissue (e.g., pancreatic adenocarcinoma cells) exposed to target specific double stranded RNA as compared to target tissues that have not been exposed to target specific double stranded RNA. Accordingly, in some embodiments, the present invention provides isolated RNA molecules (double-stranded or single-stranded) that are complementary to sequences required for pancreatic adenocarcinoma cell viability and/or replication.

### VIII. Pharmaceutical Compositions

While it is possible to administer one or both of a Wnt pathway inhibitor and a Hedgehog pathway inhibitor alone, it is preferable to administer one or both of a Wnt pathway inhibitor and a Hedgehog pathway inhibitor as a pharmaceutical formulation (composition). The inhibitors of the present invention are formulated as appropriate for use in human or veterinary medicine. In some embodiments, the inhibitors are active as given, while in other embodiments the inhibitors are provided in the form of a prodrug (e.g., compound converted into an active form in a physiological setting).

The present invention provides compositions comprising one or both of a Wnt pathway inhibitor and a Hedgehog pathway inhibitor (in a therapeutically effective amount) formulated in a pharmaceutically acceptable carrier and/or diluent. As described herein, the pharmaceutical compositions of the present invention are formulated for administration in solid or liquid form, including forms adapted for the following: (1) oral administration, for example as suspensions, tablets, boluses, powders, granules, or pastes, for application to the tongue; or (2) parenteral administration, for example as subcutaneous, intramuscular or intravenous injections in a sterile solution or suspension. However, in certain embodiments the subject compounds may be simply dissolved or suspended in sterile water. In preferred embodiments, the pharmaceutical preparation is non-pyrogenic (e.g., does not elevate the body temperature of a subject).

Pharmaceutically acceptable carriers of the present invention include pharmaceutically acceptable materials, compositions or vehicles, such as a liquid or solid filler, diluent, excipient, solvent or encapsulating material, involved in carrying or transporting the Wnt and Hedgehog pathway inhibitors from one organ, or portion of the body, to another organ, or portion of the body. Each carrier is acceptable in the sense of being compatible with the other ingredients of the formulation and not injurious to the mammalian subject. Some examples of suitable pharmaceutically acceptable carriers include but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, the Wnt and/or Hedgehog pathway inhibitors contain a basic functional group, such as amino or alkylamino, and are, thus, capable of forming pharmaceutically acceptable salts with pharmaceutically acceptable acids. Pharmaceutically acceptable salts in this respect, refers to the relatively non-toxic, inorganic and organic acid addition salts of compounds of the present invention. These salts are prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts and the like. (See, Berge et al. J Pharm Sci, 66:1-19, 1977). The pharmaceutically acceptable salts of the Wnt and Hedgehog pathway inhibitors of the present invention include the conventional nontoxic salts or quaternary ammonium salts of the compounds (e.g., from non-toxic organic or inorganic acids). For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In alternative embodiments, the Wnt and Hedgehog pathway inhibitors of the present invention contain one or more acidic functional groups and, thus, are capable of forming pharmaceutically acceptable salts with pharmaceutically acceptable bases. Pharmaceutically acceptable salts include the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (Berge et al., supra, 1977).

Formulations of the present invention include those suitable for oral and/or parenteral administration. The formulations are conveniently presented in unit dosage form and are be prepared by any methods well known in the art of pharmacy. The amount of active ingredient, which can be combined with a carrier material to produce a single dosage form will vary depending upon the subject being treated and the particular mode of administration. The amount of active ingredient combined with a carrier material to produce a single dosage form is generally that amount of the compound that produces a therapeutic effect. Generally, out of one hundred percent, this amount will range from about 1% to about 99% of active ingredient, preferably from about 5% to about 70%, most preferably from about 10% to about 30%.

Formulations of the invention suitable for oral administration are in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention can also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions also comprise buffering agents. Solid compositions of a similar type are also be employed as fillers in soft and hard-filled gelatin capsules using such excipients as lactose or milk sugars, as well as high molecular weight polyethylene glycols and the like.

A tablet is made by compression or molding, optionally with one or more accessory ingredients. Compressed tablets are be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets are produced by molding a mixture of the powdered compound moistened with an inert liquid diluent in a suitable machine. The tablets, and other solid dosage forms of the pharmaceutical compositions of the present invention, such as dragees, capsules, pills and granules, are optionally scored or prepared with coatings and shells, such as enteric coatings and other coatings well known in the pharmaceutical-formulating art. They are also formulated so as to provide slow or controlled release of the active ingredient therein using, for example, hydroxypropylmethyl cellulose in varying proportions to provide the desired release profile, other polymer matrices, liposomes and/or microspheres. They are sterilized by, for example, filtration through a bacteria-retaining filter, or by incorporating sterilizing agents in the form of sterile solid compositions that can be dissolved in sterile water, or some other sterile injectable medium immediately before use. These compositions also optionally contain opacifying agents and are of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in a microencapsulated form, if appropriate, with one or more excipients.

Liquid dosage forms for oral administration of the compounds of the invention include pharmaceutically acceptable emulsions, microemulsions, solutions, suspensions, syrups and elixirs. In addition to the active ingredient, the liquid dosage forms optionally contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

It is known that sterols, such as cholesterol, form complexes with cyclodextrins. Thus, in some preferred embodiments, where the inhibitor is a steroidal alkaloid (e.g., cyclopamine), it is formulated with cyclodextrins, such as alpha-, beta- and gamma-cyclodextrin, dimethyl-beta cyclodextrin and 2-hydroxypropyl-beta-cyclodextrin.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which are reconstituted into sterile injectable solutions or dispersions just prior to use, and which optionally contain antioxidants, buffers, bacteriostats, solutes (rendering the formulation isotonic with the blood of the intended recipient) or suspending or thickening agents. Examples of suitable aqueous and nonaqueous carriers that are employed in some pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity is maintained, for example, by the use of coating materials, such as lecithin, by the maintenance of the required particle size in the case of dispersions, and by the use of surfactants. These compositions may also contain adjuvants such as preservatives, wetting agents, emulsifying agents and dispersing agents. Prevention of the action of microorganisms is ensured by the inclusion of various antibacterial and antifungal agents, for example, paraben, chlorobutanol, phenol sorbic acid, and the like. It is also be desirable in some embodiments to include isotonic agents, such as sugars, sodium chloride, and the like into the compositions. In addition, prolonged absorption of the injectable pharmaceutical form can be brought about by the inclusion of agents that delay absorption, such as aluminum monostearate and gelatin.

In some cases, in order to prolong the effect of a drug, it is desirable to slow the absorption of the drug from subcutaneous or intramuscular injection. This can be accomplished by the use of a liquid suspension of crystalline or amorphous material having poor water solubility. The rate of absorption of the drug then depends upon its rate of dissolution, which, in turn, depends upon crystal size and crystalline form. Alternatively, the delayed absorption of a parenterally administered drug, is accomplished by dissolving or suspending the drug in an oil vehicle. Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

A therapeutically effective amount or dose is that amount which is effective for producing a desired therapeutic effect by inhibiting Wnt and Hedgehog activity in at least a sub-population of cells in a subject and thereby blocking the undesirable biological consequences of these pathways (e.g., dysregulated growth) in the treated cells, at a reasonable benefit/risk ratio applicable to any medical treatment. Toxicity and therapeutic efficacy of such compounds are determined by standard pharmaceutical procedures in cell cultures or experimental animals. Briefly, the LD₅₀ is the dose lethal to 50% of the study population, while the ED₅₀ is the dose therapeutically effective in 50% of the study population. The dose ratio between toxic and therapeutic effects is the therapeutic index, which can be expressed as the ratio LD₅₀/ED₅₀. Compounds exhibiting large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that can be taken into account include the severity of the disease state; age, weight, and gender of the patient; diet, time and frequency of administration, drug combination(s), reaction sensitivities, and tolerance/response to therapy. Long acting pharmaceutical compositions are typically administered every 3 to 4 days, every week, or once every two weeks depending on the half life and clearance rate of the particular formulation. Normal dosage amounts may vary from 0.1 to 100,000 micrograms, up to a total dose of about 1 gram, depending upon the route of administration. Guidance as to particular dosages and methods of delivery is provided in the literature (See, U.S. Pat. Nos. 4,657,760; 5,206,344; or 5,225,212; all of which are herein incorporated by reference).

### IX. Screening Tumor Cells For Wnt and Hedgehog Pathway Activity

The present invention also provides methods for determining whether one or both of the Wnt and Hedgehog signaling pathways are active in a cell of interest. In preferred embodiments, these methods are suitable for screening tumor biopsy specimens to determine whether treatment of the tumor with Wnt and Hedgehog pathway inhibitors is advisable. Specifically, tumor cells with elevated expression (as compared to untransformed cells of the same tissue) of two or more of the Hedgehog pathway components selected from Patched, Smoothened, Gli1 and Gli2, are contemplated to be suitable candidates for treatment with a Hedgehog pathway inhibitor. Likewise, tumor cells with elevated expression (as compared to untransformed cells of the same tissue) of the Wnt pathway component β-catenin, in the presence or absence of elevated expression of Lef1 and Tcf, are contemplated to be suitable candidates for treatment with a Wnt pathway inhibitor. Methods for measurement of elevated expression of signaling pathway components include for example RT-PCR. Other suitable methods for measuring differences in RNA or protein expression include but are not limited to microarray analysis and immunohistochemistry.

### Experimental

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); and C (degrees Centigrade).

### EXAMPLE 1

### Immunohistochemistry, Microscopy and Flow Cytometry

Immunohistochemical and immunofluorescence analyses were performed on paraffin sections as described previously (Kim et al., Development, 124:4243-4252, 1997; and Hebrok et al., Development, 127:4905-4913, 2000). Briefly, paraffin-embedded tissues were cut into 5-6 μm thick sections, deparaffinized in xylene, and hydrated in decreasing concentrations of ethanol. Slides were washed in PBS and used either for hematoxylin/eosin, immunohistochemistry (DAB staining), or immunofluorescence analysis. Mouse anti β-catenin antibody (BD Biosciences) and mouse anti-cleaved caspase 3 antibody (Cell Signaling Technology) were both used at a 1:100 dilution. The Alexa488-conjugated anti-mouse polyclonal (Molecular Probes) was used at a 1:300 dilution as a secondary antibody for immunofluorescence. Alternatively, the biotinylated anti-goat IgG (Vector) was used at a 1:200 dilution as a secondary antibody. Staining for diaminobenzidine (DAB) was performed with the ABC Elite immunoperoxidase system according to manufacturer's instructions (Vector). Fluorescence was visualized and photographed with a Zeiss Axioskop2 plus microscope.

For flow cytometric analysis of cleaved caspase 3 expression, cells were harvested and fixed in 2% formaldehyde at 37° C. for 10 min. Subsequently, cells were permeabilized in 90% ice-cold methanol (30 min on ice) and then resuspended in PBS with 0.5% BSA. The primary anti-cleaved caspase 3 antibody was used at a 1:25 dilution, while the secondary Alexa488-conjugated anti-mouse antibody was used at a 1:200 dilution.

### EXAMPLE 2

### Cell Culture

Human pancreatic adenocarcinoma cell lines HPAC, SW1990, Mpanc-96, SU86.86, PL45, Panc10.05 Panc2.03 and Panc8.13 were obtained from ATCC; cell lines MiaPaCa2, Panc1, CFPAC-1, HPAFII, Capan-2, AsPC1, Hs766T and BxPC3 were a gift from Schering Plough; cell lines COLO357, L3.3, L3.6 pl and L3.6 sl were a gift from I. Fidler; cell lines Panc3.27, Panc4.21, Panc5.04, Panc2.13, Panc1.28 and Panc6.03 were a gift from E. Jaffee. BxPC3 and the Panc cell lines were grown in RPMI medium (Gibco) supplemented with 10% FBS, L-glutamine and penicillin/streptomycin; medium for the Panc cells was also supplemented with insulin-transferrin-selenium (Gibco). The CFPAC-1, Panc1, AsPC1, Hs766T, L3.6sl and L3.6pl were grown in DMEM without phenol red (CellGro), supplemented with 10% FBS. Derivation and culture of PDC cells was as described (Schreiber et al., Gastroenterology, 127:250-260, 2004).

### EXAMPLE 3

### Drug Treatment

The small chemical inhibitors were used at the following final concentrations: up to 250 μM for sulindac (Sigma), up to 300 μM for sulindac sulfone (Calbiochem or ICN), NS-398 up to 25 μM (Calbiochem), sodium salicylate up to 100 μM (Calbiochem). Cyclopamine (Toronto Research Chemicals) was used at 10 μM. To test for drug sensitivity, the cells were plated in control medium or in medium containing the drug of interest for 2 to 5 days. The culture media was changed every 48 hrs. The images showing cell morphology were taken with a Nikon inverted microscope.

### EXAMPLE 4

### BrdU Incorporation Assay

Cells were pulsed with 10 μM BrdU for two hours and then harvested and fixed. BrdU incorporation was detected with a fluorescein isothiocyanate conjugated anti-BrdU antibody (BD Biosciences) and total DNA was stained with 7-AAD (BD Biosciences). FACS analysis was performed according to the BD Biosciences BrdU flow kit manual. Cells in S-phase were defined as the BrdU-positive cell population with a DNA content between 2N and 4N. According to the manual, apoptotic cells were defined as a subpopulation of G₀/G₁ cells with DNA content lower than the diploid amount (<2N).

### EXAMPLE 5

### Transient and Stable Transfections

The cell lines were plated the day prior to transfection at a density of 8×10⁴ cells/well in a 24-well plate. The luciferase reporter constructs used included: Top-Flash and Fop-Flash containing multimerized Tcf-Lef binding sites or nonfunctional sites, respectively; TK-Luc (Promega); and GliBS containing 8×Gli binding sites upstream of the TK minimal promoter (Sasaki et al., Development, 124:1313-1322, 1997). In different experiments, the reporter constructs were co-transfected with an expression vector containing a dominant-negative form of Lef1 or the endogenous Wnt inhibitor Icat. The dominant negative Lef1 (gift from S. Pleasure) was generated by cloning the 63 amino acid N-terminal deleted form of Lef1 into the pEF5 plasmid. This construct also contains an IRES-EGFP cassette. The cDNA coding for the Icat protein was cloned into pIRES2-EGFP (BD Biosciences Clontech). Transfections were performed using the Effectene transfection reagent (Promega) according to the user's manual. A Renilla luciferase plasmid (Promega) was included to control for transfection efficiency. Transfected cells were cultured in medium containing serum. In the experimental samples, the final concentration used for sulindac was 250 μM, for sulindac sulfone of 150 μM, and for cyclopamine of 10 μM. Cells were harvested 24 hrs after transfection, and luciferase activity was assayed using the Dual Luciferase kit (Promega), according to the user's manual.

For each Fop-luciferase or Top-luciferase experiment, a renilla luciferase construct was included. For each experimental condition (drug treatment, co-transfection or siRNA), triplicate Top- and Fop-luciferase samples were analyzed. To equilibrate the total vector-DNA (or siRNA) amount in all samples, equivalent amounts of an empty expression vector or unrelated siRNAs were transfected into control samples. The following equations were used to obtain the normalized luciferase values and the relative (fold) activation. The firefly luciferase (Fop and Top) values were divided by the renilla luciferase values to obtain the normalized luciferase values. The normalized Top values for each condition were divided by the Fop values of the same condition (e.g., untreated Top/untreated Fop; drug-treated Top/drug-treated Fop). As expected, the Fop values remained constant under the different conditions. To minimize the number of bars in each figure, only representative average Fop values are included in the graphs.

For stable transfections, the dominant active form of Lef1 was generated by cloning the N-terminal deleted form (63 amino acid) of Lef1 in operable combination with a VP16 activation domain as a fusion protein in the pEF5 plasmid. The construct also contains an IRES-EGFP cassette. After transfection, PDCs were grown in medium containing 300 μg/ml G418 for three weeks. Surviving cells were subsequently pooled and maintained as a stable line.

### EXAMPLE 6

### RT-PCR ANALYSIS

RNA from pancreatic cancer cells and PDCs was prepared using TRIzol reagent (Invitrogen). Approximately 2 μg of total RNA was treated with DNAse RQ1 (Promega) prior to cDNA synthesis using random hexamers and Superscript II reverse transcriptase (Invitrogen). The oligonucleotides used for gene-specific amplification of the human sequences are provided in Table 4, while those used for amplification of the mouse sequences are provided in Table 5.

TABLE 4

Oligonucleotides Used For
Amplification of Human Sequences

| | | |
|---|---|---|
| Primer | Sequence | Identifier |

| | | |
|---|---|---|
| Wnt11 sense | CACTGAACCAGACGCAACA | SEQ ID NO:1 |
| | C | |
| Wnt11 antisense | ATACACGAAGGCCGACTCC | SEQ ID NO:2 |
| Wnt7b sense | GGGACTATGAACCGGAAAG | SEQ ID NO:3 |
| | C | |
| Wnt7b antisense | TGCGGAACTGAAACTGACA | SEQ ID NO:4 |
| | C | |
| Wnt5b sense | TCTGACAGACGCCAACTCC | SEQ ID NO:5 |
| | T | |
| Wnt5b antisense | GCATTCCTTGATGCCAGTC | SEQ ID NO:6 |
| | T | |
| Wnt2b sense | AGACATCATGCGTTCAGTG | SEQ ID NO:7 |
| | G | |
| Wnt2b antisense | TCACAGCTGCACACACTCA | SEQ ID NO:8 |
| | G | |
| Tcf1 sense | CCTTGATGCTAGGTTCTGG | SEQ ID NO:9 |
| | TG | |
| Tcf1 antisense | GCAGCGCTCTCCTTAAGTG | SEQ ID NO:10 |
| | T | |
| Tcf3 sense | CGGTCCTCTCCTCACATGG | SEQ ID NO:11 |
| | T | |
| Tcf3 antisense | TGCCACTTTCTTCCAAGGA | SEQ ID NO:12 |
| | T | |
| Tcf4 sense | CATATGGTCCCACCACATC | SEQ ID NO:13 |
| | A | |
| Tcf4 antisense | TGCTCTTCTCTGGACAGTG | SEQ ID NO:14 |
| | C | |
| Lef1 sense | TCATATGATTCCCGGTCCT | SEQ ID NO:15 |
| | C | |
| Lef1 antisense | CCTTCTGCCAAGAATCTGG | SEQ ID NO:16 |
| | T | |
| Lrp5 sense | CTCTCCGGGGACACTCTGT | SEQ ID NO:17 |
| | A | |
| Lrp5 antisense | CTCCTGCCTTACACGTCCT | SEQ ID NO:18 |
| | G | |
| Lrp6 sense | ACAGACACTGGCACTGATC | SEQ ID NO:19 |
| | G | |
| Lrp6 antisense | GTTTTGGCATCTCCCCAGT | SEQ ID NO:20 |
| | A | |
| COX-2 sense | CAGCACTTCACGCATCAGT | SEQ ID NO:21 |
| | T | |
| COX-2 antisense | CAGCAAACCGTAGATGCTC | SEQ ID NO:22 |
| | A | |
| β-actin-sense | GCATTGTTACAGGAAGTC | SEQ ID NO:23 |
| | CCTTGCC | |
| β-actin-antisense | ACCCACTCCCAGGGAGAC | SEQ ID NO:24 |
| | CAAAAG | |

TABLE 5

Oligonucleotides Used For
Amplification of Mouse Sequences

| | | |
|---|---|---|
| Primer | Sequence | Identifier |

| | | |
|---|---|---|
| CK19 sense | GAAGAAGAACCATGAGGAGGA | SEQ ID NO:25 |
| CK19 antisense | AAGTCGAGGGAGGGGTTAGAG | SEQ ID NO:26 |
| CFTR sense | CTTTCGGAGTGATAACACAGA | SEQ ID NO:27 |
| CFTR antisense | AGGCTTGTGCTTGCTGGAG | SEQ ID NO:28 |
| Mud sense | TCTATTTCCTTGCCCTGGCAG | SEQ ID NO:29 |
| Mud antisense | GTTCCCATCCCTGTCTCCAG | SEQ ID NO:30 |
| Car2 sense | ACTCTCAGGACAATGCAGTG | SEQ ID NO:31 |
| Car2 antisense | TACAGAGAGGCGGTCACAC | SEQ ID NO:32 |
| Ptc1 sense | TGTCTTTGCCCGGTCCACTG | SEQ ID NO:33 |
| Ptc1 antisense | AGGGGACAAGGAGCCAGAGT | SEQ ID NO:34 |
| Gli1 sense | CATAGGGTCTCGGGGTCTCA | SEQ ID NO:35 |
| Gli1 antisense | CCTGCGGCTGACTGTGTAAG | SEQ ID NO:36 |
| Smo sense | CAGCATGTCACCAAGATGGTG | SEQ ID NO:37 |
| | GC | |
| Smo antisense | ACAGGGGCAGAGTGGTGAAGC | SEQ ID NO:38 |
| | TCAG | |
| Shh sense | ATGCTGGCTCGCCTGGCTGTG | SEQ ID NO:39 |
| | GAA | |
| Shh antisense | TGAGGAAGTCGCTGTACAGCA | SEQ ID NO:40 |
| Wnt1 sense | CTGGGTTTCTACTACGTTGC | SEQ ID NO:41 |
| Wnt1 antisense | GTTCTGGTCGGATCAGTCG | SEQ ID NO:42 |
| Wnt2b sense | TGGAGGGCACTCTCAGACTTC | SEQ ID NO:43 |
| | C | |
| Wnt2b antisense | GCCTTGTCCAAGACACAGTAG | SEQ ID NO:44 |
| | T | |
| Wnt5a sense | CTTCCGCAAGGTGGGCGATGC | SEQ ID NO:45 |
| Wnt5a antisense | TTGCACAGGCGTCCCTGCGTG | SEQ ID NO:46 |
| Wnt11 sense | GTGGCTGCTGACCTCAAGACC | SEQ ID NO:47 |
| Wnt11 antisense | TTCTTCATGCAGAAGTCAGGA | SEQ ID NO:48 |
| | G | |
| Lef1 sense | ACAGTGACCTAATGCACGTGA | SEQ ID NO:49 |
| | AGCC | |
| Lef1 antisense | CGCTGACCAGCCTGGATAAAG | SEQ ID NO:50 |
| | CT | |
| Tcf1 sense | GAAGCCAGTCATCAAGAAACC | SEQ ID NO:51 |
| | CCTC | |
| Tcf1 antisense | TGTTTTTCCCTTGACCGCCTC | SEQ ID NO:52 |
| | TTC | |
| Tct3 sense | GAGAAGCCTTGTGATAGCCCT | SEQ ID NO:53 |
| | GCG | |
| Tcf3 antisense | AAGTAGGGGGAGAGGTCAGCA | SEQ ID NO:54 |
| | GAGC | |
| Tcf4 sense | AGAGCGAAGGTGGTGGCCGAA | SEQ ID NO:55 |
| | T | |
| Tcf4 antisense | GGAAGCGAAAGGCAAGGATTT | SEQ ID NO:56 |
| | AGG | |

### EXAMPLE 7

### Small Interfering RNA (siRNA) Treatment

Small interfering RNA (siRNA) oligonucleotides against β-catenin containing two thymidine residues (dTdT) at the 3′ end of the sequence (5′-AGCUGAUAUU GAUGGACAGT T-3′ set forth as SEQ ID NO:57) were used for treatment of cancer cells as described (Verma et al., Clin Cancer Res, 9:1291-1300, 2003). The GLI1 siRNA (sense 5′-CUCCACAGGC AUACAGGAU-3′ set forth as SEQ ID NO:58) was also used as described (Sanchez et al., Proc Natl Acad Sci USA 101:12561-12566, 2004). Small interfering RNA against GLI2 was ordered against the target sequence 5-AAACACCAAC CAGAACAAGC A-3′, set forth as SEQ ID NO:59). The siRNA oligonucleotides, as well as a fluorescent-labeled non-targeting siRNA (siGlo RISC-Free siRNA), were obtained from Dharmacon Research Inc. According to the manufacturer, the siGlo RISC-Free siRNA negative control was bioinformatically designed to have greater than four mismatches with known human and mouse genes. Cells were transfected with oligofectamine (Invitrogen) according to the user's manual, with a final siRNA concentration of 100 or 300 nM. Transfected cells were incubated for 48 hrs before harvesting.

Pancreatic cancer cell lines BxPC3, CFPAC and Panc4.21 were co-transfected with the GLI reporter vector GliBS-LUC or with the control vector TK-LUC and a control siRNA or a combination of GLI1 and GLI2 siRNA. Simultaneous targeting of GLI1 and GLI2 resulted in significant (e.g., 40-70%) reduction in Hedgehog activity as measured by the GliBS-luciferase assay.

### EXAMPLE 8

### Allograft Treatment In Vivo

A total of 0.1 ml Hanks' balanced salt solution and matrigel (1:1) containing 2×10⁶ cells was injected subcutaneously into CD-1 nude mice (Charles River Laboratories). Tumors were grown for 4 days to a minimum volume of 125 mm³. Treatment was initiated simultaneously for all subjects. Mice were injected with vector alone (peanut oil: PBS 7:3 v/v) or with a sulindac solution (3 mg/mouse/day) daily for 5 days. Sulindac sulfone was administered orally (1.5 mg/mouse/day). At the end of the treatment period, tumors were excised from mice, weighed and photographed.

To examine the efficacy of simultaneous cyclopamine/sulindac sulfone treatment to block tumor growth in vivo, L3.6sl cells were injected subcutaneously. Individual or combined treatment was initiated after tumors were palpable. Sulindac sulfone and cyclopamine were used at half the amount that has shown efficacy in preventing tumor growth when used as single agents: 0.75 mg/animal/day for sulindac sulfone and 0.5 mg/animal/day for cyclopamine. However, the present invention is not limited to these concentrations, and both higher and lower concentrations of sulindac sulfone and cyclopamine are contemplated to find use with the methods and compositions of the present invention.

A reduction in L3.6sl tumor size was observed in animals treated with sulindac sulfone plus cyclopamine, as compared to tumor size in untreated animals, and in animals treated with sulindac sulfone or cyclopamine alone. In addition, the weight of the tumor in animals treated with both sulindac sulfone and cyclopamine was significantly lower (p<0.01) than the tumors in untreated animals and in animals treated with sulindac sulfone alone or cyclopamine alone. Thus as described herein for the first time, simultaneous inhibition of Hedgehog and Wnt signaling pathways blocks cancer cell growth in vivo.

### EXAMPLE 9

### Soft Agar Assay

Cells were plated in 6-well plates in respective culture medium supplemented with 0.2% agarose. Liquid media was added to each well, and changed every 48 hrs. The cells were kept in culture for up to 8 weeks.

### EXAMPLE 10

### Western Blot Analysis

Protein extracts were prepared by lysing cells on ice for 30 minutes using RIPA buffer (150 mM NaCl, 0.5% sodium deoxycholate, 0.1% SDS, 50 mM Tris-HCl [pH 7.5], 1% Triton X-100, 2X complete protease inhibitors—EDTA free [Roche Molecular Biochemicals]). Lysates were cleared by centrifugation and concentrations of soluble extract were normalized using Bradford reagent (Biorad). Extracts were fractionated on a 10% SDS-polyacrylamide gel (Biorad Ready Gel), and transferred to a nitrocellulose membrane (Biorad Transblot Transfer medium) using standard procedures. Membranes were proved with an anti-dephospho β-catenin antibody (Alexis) at a 1:100 dilution and an anti-GAPDH polyclonal rabbit antibody (Santa Cruz) at a 1:100 dilution.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in the relevant fields, are intended to be within the scope of the following claims.

We claim:

1. A method comprising contacting a pancreatic adenocarcinoma cell with both a Hedgehog pathway inhibitor and a Wnt pathway inhibitor, under conditions suitable for retarding growth of said pancreatic adenocarcinoma cell.

2. The method of claim 1, wherein said pancreatic adenocarcinoma cell is a human cell.

3. The method of claim 1, wherein said retarding growth comprises inhibiting proliferation of said pancreatic adenocarcinoma cell.

4. The method of claim 1, wherein said retarding growth comprises inducing death of said pancreatic adenocarcinoma cell.

5. The method of claim 4, wherein said inducing death of said pancreatic adenocarcinoma cell is essentially unaccompanied by death of untransformed pancreatic ductal cells.

6. The method of claim 1, wherein said Hedgehog pathway inhibitor is cyclopamine or Gli siRNA.

7. The method of claim 1, wherein the target of said Hedgehog pathway inhibitor is selected from the group consisting of Smoothened, Sonic Hedgehog, Desert Hedgehog, Indian Hedgehog, Gli1 and Gli2.

8. The method of claim 1, wherein said Wnt pathway inhibitor is selected from the group consisting of sulindac, sulindac sulfide, sulindac sulfone, ICAT, dnLef1, and β-catenin siRNA.

9. The method of claim 1, wherein the target of said Wnt pathway inhibitor is selected from the group consisting of LRP, Frizzled, Wnt, Dishevelled, β-catenin, Lef and Tcf.

10. The method of claim 1, further comprising assessing Wnt pathway activity in said adenocarcinoma cell by examining nuclear or cytoplasmic accumulation of β-catenin, prior to said contacting.

11. A method of treating cancer of the pancreas, comprising:
a) providing a Hedgehog pathway inhibitor and a Wnt pathway inhibitor; and
b) administering said Hedgehog pathway inhibitor and said Wnt pathway inhibitor to a subject diagnosed with cancer of the pancreas, under conditions suitable for retarding growth of said cancer.

12. The method of claim 11, wherein said subject is a human.

13. The method of claim 11, wherein said cancer of the pancreas is pancreatic adenocarcinoma.

14. The method of claim 13, wherein said retarding growth of said cancer comprises inhibiting proliferation of at least one cell of said pancreatic adenocarcinoma.

15. The method of claim 13, wherein said retarding growth of said cancer comprises inducing death of at least one cell of said pancreatic adenocarcinoma.

16. The method of claim 15, wherein said inducing death of at least one cell of said pancreatic adenocarcinoma is essentially unaccompanied by death of untransformed pancreatic ductal cells.

17. The method of claim 13, wherein said retarding growth of said cancer comprises reducing volume of said pancreatic adenocarcinoma.

18. The method of claim 11, wherein said Hedgehog pathway inhibitor and said Wnt pathway inhibitor are each formulated in a pharmaceutically acceptable carrier.

19. The method of claim 11, further comprising administering a standard treatment regimen for cancer of the pancreas, to said subject.

20. The method of claim 19, wherein said standard treatment regimen comprises one or more of resection, radiation and chemotherapy.

21. The method of claim 20, wherein said chemotherapy is selected from the group consisting of 5-fluorouracil, and 2′,2′-difluorodeoxycytidine.

22. The method of claim 11, further comprising administering an adjunct treatment regimen for cancer of the pancreas, to said subject.

23. The method of claim 22, wherein said adjunct treatment regimen is selected from the group consisting of a platinum analogue, topoisomerase-inhibitor, antimicrotubule agent, proteosome inhibitor, vitamin D analogue, arachidonic acid pathway inhibitor, histone deacytylator inhibitor, farnesyltransferase inhibitor and epidermal growth factor-based therapy.

24. A kit for treating pancreatic adenocarcinoma in a subject, comprising: a) a Hedgehog pathway inhibitor; b) a Wnt pathway inhibitor; and c) instructions for using said Hedgehog pathway inhibitor and said Wnt pathway inhibitor to treat pancreatic adenocarcinoma in a subject.

25. The kit of claim 24, wherein said Hedgehog pathway inhibitor is cyclopamine or Gli siRNA.

26. The kit of claim 24, wherein said Wnt pathway inhibitor is selected from the group consisting of sulindac, sulindac sulfide, sulindac sulfone, ICAT, dnLef1, and β-catenin siRNA.
